(19) United States
(12) Patent Application Publication
(10) Pub. No.: US 2023/0374177 A1
(43) Pub. Date: Nov. 23, 2023

(54) CYTOSOLIC MICROPARTICLES, PHAGOCYTIC CELLS COMPRISING THE SAME, AND METHODS FOR TREATING DISEASE COMPRISING THE SAME

(71) Applicant: Florida State University Research Foundation, Inc., Tallahassee, FL (US)

(72) Inventors: Wenhao CHENG, Tallahassee, FL (US); Jingjiao GUAN, Tallahassee, FL (US)

(21) Appl. No.: 18/320,272

(22) Filed: May 19, 2023

Related U.S. Application Data
(60) Provisional application No. 63/365,036, filed on May 20, 2022.

(51) Int. Cl. C08F 220/54; B82Y 5/00; A61P 35/00; A61K 35/17; A61K 33/26; A61K 31/715

(52) U.S. Cl. CPC C08F 220/54; B82Y 5/00; A61P 35/00; A61K 35/17; A61K 33/26; A61K 31/715

(57) ABSTRACT

In one aspect, the disclosure relates to cytosolic microparticles comprising a polymeric material such as, for example, poly(N-isopropylacrylamide) (PNIPAM), and containing nanoparticles, and methods for producing the same. The disclosure further relates to phagocytic cells such as macrophages containing the cytosolic microparticles, wherein the microparticles are not subject to the harsh environment of the phagosome. Also disclosed are compositions containing the phagocytic cells and methods for treating diseases in a subject, including various cancers, wherein the methods include administering the phagocytic cells or disclosed compositions to a subject.

### CROSS-REFERENCE TO RELATED APPLICATIONS

This application claims the benefit of U.S. Provisional Application No. 63/365,036, filed on May 20, 2022, which is incorporated herein by reference in its entirety.

### STATEMENT REGARDING FEDERALLY SPONSORED RESEARCH OR DEVELOPMENT

This invention was made with government support under grant number 1R03CA202334-01A1 awarded by the National Cancer Institute of the National Institutes of Health, grant number R03EB028878 awarded by the National Institute of Biomedical Imaging and Bioengineering of the National Institutes of Health, and grant number 1661727 awarded by the National Science Foundation. The government has certain rights in the invention.

### BACKGROUND

Solid-tumor-based cancer is a major cause of death in the United states, yet no effective therapy exists for treating metastatic solid tumors. Adoptive macrophage therapy for treating cancer previously attracted tremendous interest and was evaluated by clinical trials from 1987 to 2010 without success. The recent success of chimeric antigen receptor (CAR) T-cell therapy for treating hematologic cancers and its inability to treat solid tumors has reignited the interest in the use of the adoptive macrophage therapy for treating solid tumors, because macrophages can naturally accumulate in the solid tumors. However, macrophages in the tumors are typically polarized by the tumor microenvironment to a phenotype that promotes tumor progression. It is thus believed that the adoptive macrophages in the solid tumors need to maintain a cancer-fighting phenotype in order to be effective in cancer treatment.

Two major methods are currently known for keeping the adoptive macrophages in the cancer-fighting phenotype in the solid tumors. The first method relies on attaching drug-loaded microparticles to the exterior of the macrophages. The drug can be released from the microparticles to keep the macrophages in the cancer-fighting phenotype. The other method relies on the genetic modification of macrophages with viral vectors. Both methods are at the preclinical stages of development. However, the drugs released in the first method may still be exposed to a harsh environment in the extracellular fluid or in phagosomes if taken up by the macrophages following release, and the second method may still require the use of high doses of chemotherapeutic agents, which can have systemic side effects in patients, or DNA or RNA of viral origin may be found to trigger an immune response.

Macrophage phagocytosis is characterized by the internalization of an object larger than 0.5 μm in diameter into a membrane-bound vacuole known as a phagosome. Examples of such objects are inorganic particles, live bacteria, and cancer cells. Phagosomal rupture, which refers to the rupture of a phagosome, plays a critical role in the development of silicosis, in the virulence of infectious microorganisms, such as Mycobacterium tuberculosis and Listeria monocytogenes, as well as in the establishment of acquired immunity against tumors and viruses. The molecular mechanisms that cause phagosomal rupture in response to various phagocytic objects are not yet fully understood, mainly because these objects are highly complex in structure and composition.

Despite advances in adoptive macrophage therapy research, there is still a scarcity of methods that serve to deliver therapeutic nanoparticles to the interior of phagocytic cells while also protecting the nanoparticles from the harsh environment of the phagosome and while allowing the macrophages or other phagocytic cells to retain a cancer-fighting phenotype. An ideal method would have a predictable outcome based on an understanding of the molecular mechanisms that cause phagocytic rupture. These needs and other needs are satisfied by the present disclosure.

### SUMMARY

In accordance with the purpose(s) of the present disclosure, as embodied and broadly described herein, the disclosure, in one aspect, relates to cytosolic microparticles comprising a polymeric material such as, for example, poly(N-isopropylacrylamide) (PNIPAM), and containing nanoparticles, and methods for producing the same. The disclosure further relates to phagocytic cells such as macrophages containing the cytosolic microparticles, wherein the microparticles are not subject to the harsh environment of the phagosome. Also disclosed are compositions containing the phagocytic cells and methods for treating diseases in a subject, including various cancers, wherein the methods include administering the phagocytic cells or disclosed compositions to a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a schematic representation of inducing phagosomal rupture in a live macrophage using a PNIPAM microparticle. “Nu.” represents the nucleus.

FIGS. 2A(i)-2E(iv) show characterization of PNIPAM microparticles labeled with fluorescent nanoparticles. (FIGS. 2A(i)-2A(iii)) Bright-field image (FIG. 2A(i)), fluorescence image (FIG. 2A(ii)), and merged image (FIG. 2A(iii)) of the microparticles printed on a glass coverslip. (FIGS. 2B(i)-2B(ii)) SEM images of the microparticles printed on a glass coverslip. (FIG. 2C(i)) Merged bright-field and fluorescence image of a cutting edge of a PVA film carrying the microparticles. A microparticle that was cut into half is indicated by an arrow. (FIG. 2C(ii)) Fluorescence image of the cross section of a cutting edge of a PVA film carrying the microparticles. Two microparticles are indicated by arrows. (FIGS. 2D(i)-2D(iii)) Bright-field image (FIG. 2D(i)), fluorescence image (FIG. 2D(ii)), and merged image (FIG. 2D(iii)) of microparticles that had been soaked in 37° C. PBS for 7 d. Objects in (FIG. 2D(iii)) are not completely overlapped because the microparticles were moving when imaged. (FIG. 2E(i)) Time course of temperature of the complete medium in which the microparticles were soaked during imaging. (FIGS. 2E(ii)-2E(iv)) Fluorescence images of the microparticles in the complete medium at (FIG. 2E(ii)) 35.6, (FIG. 2E(iii)) 29, and (FIG. 2E(iv)) 28.5° C. The microparticles were bright and relatively small at 35.6° C., swelled and dimmed at 29° C., and completely disappeared at 28.5° C. Scale bar in (FIG. 2B(ii)) represents 5 μm, and scale bars in all other images represent 20 μm.

FIGS. 3A(i)-3C(ii) show phagosomal rupture in macrophages caused by PNIPAM or PNIPAM-fluorescein microparticles. The temperature or temperature range marked on an image indicates the estimated temperature or temperature range at which the image was taken. (FIGS. 3A(i)-3A(iv)) Merged bright-field, nanoparticle fluorescence, and Hoechst 33342 fluorescence images of macrophages and microparticles labeled with fluorescent nanoparticles. (FIG. 3A(i)) was taken after the non-phagocytosed microparticles dissolved in an ambient environment. (FIG. 3A(ii)) is a magnified view of the microparticle marked by “a” in (FIG. 3A(i)). (FIG. 3A(iii)) was taken after a 0° C. cold shock. (FIG. 3A(iv)) is a magnified view of the microparticle marked by “a” in (FIG. 3A(iii)). (FIGS. 3B(i)-3B(ii)) Merged bright-field, nanoparticle fluorescence, and LysoView 488 fluorescence images of macrophages and microparticles labeled with fluorescent nanoparticles. (FIG. 3B(i)) was taken after the culture was washed with and incubated in 22° C. PBS. (FIG. 3B(ii)) was taken after a 0° C. cold shock. (FIGS. 3C(i)-3C(ii)) Merged bright-field and PNIPAM-fluorescein fluorescence images of macrophages and microparticles. (FIG. 3C(i)) was taken after the culture was washed with and incubated in 37° C. PBS. (FIG. 3C(ii)) was taken after a 0° C. cold shock. Scale bars in (FIGS. 3A(i), 3A(iii), and 3B(i)-3C(ii)) represent 20 μm and those in (A2, A4) represent 10 μm.

FIG. 4 shows the effect of cold-shock temperature on the percentage of phagosomal rupture. Data presented as mean±SEM.

FIGS. 5A-5B show theoretical analysis of phagosomal rupture caused by the PNIPAM microparticles. (FIG. 5A) Model-predicted osmotic pressure of osmotic pressure difference vs radius of the microparticle/phagosome at 0° C. (FIG. 5B) Model-predicted membrane surface tension of a swelling phagosome vs radius of the microparticle/phagosome at different cold-shock temperatures.

FIG. 6 shows the effect of hypotonic shock, chloroquine, tetrandrine, colchicine, and LLOMe on the percentage of phagosomal rupture at 22° C. compared to the control treatment, which is the 22° C. cold shock. Data presented as mean±SEM.

FIG. 7 shows characterization of PNIPAM microparticles and macrophages 3 h after adding the macrophages to the microparticles. Merged bright-field, nanoparticle fluorescence, and Hoechst 33342 fluorescence image of macrophages and microparticles labelled with fluorescent nanoparticles before the non-phagocytosed microparticles dissolved. Microparticles that were clearly colocalized with the macrophages are indicated by arrows. An estimated temperature range for the complete medium is marked at the upper region of the image. Scale bar represents 10 μm.

FIGS. 8A-8B show phagosomal rupture caused by PNIPAM microparticles. Merged bright-field and nanoparticle-fluorescence images of macrophages and PNIPAM microparticles at the same area (FIG. 8A) before and (FIG. 8B) after rupture. Seven microparticle-containing macrophages in (FIG. 8A) (indicated by arrows) ruptured in (FIG. 8B) (indicated by arrows). Scale bar in (FIG. 8B) represents 20 μm and applies to both images.

FIGS. 9A-9B show phagosomal rupture caused by PNIPAM-fluorescein microparticles. (FIGS. 9A-9B) Merged bright-field and fluorescein-fluorescence images of macrophages and microparticles after a 0° C. cold shock following a 24 h-incubation at 37° C. Scale bar in (FIG. 9B) represents 20 μm and applies to both images.

FIGS. 10A-10B show the effect of post-phagocytosis time on phagosomal rupture. Merged bright-field and nanoparticle-fluorescence images of macrophages and PNIPAM microparticles after (FIG. 10A) a 3 h-incubation at 37° C. and three rinses with 22° C. PBS, and (FIG. 10B) a further 24 h-incubation at 37° C. and a 0° C. cold shock. All microparticle-containing phagosomes in (FIG. 10A) were not ruptured except the two indicated by the arrows. All microparticle-containing phagosomes in (FIG. 10B) were ruptured. Scale bar in (FIG. 10B) represents 50 μm and applies to both images.

FIGS. 11A-11B show mRNA expression levels of iNOS, IL-6 and TNF-α in the macrophages treated with LPS (1 μg/mL) for (FIG. 11A) 3 h and (FIG. 11B) 24 h assessed by real time qRT-PCR. Data presented as mean±SEM.

FIGS. 12A-12F show the effect of hypotonic shock, chloroquine, tetrandrine, colchicine and LLOMe on phagosomal rupture at 22° C. compared to the control, which is the 22° C. cold shock. Merged bright-field and nanoparticle-fluorescence images of macrophages and PNIPAM microparticles treated with (FIG. 12A) the control condition, (FIG. 12B) hypotonic shock, (FIG. 12C) chloroquine, (FIG. 12D) tetrandrine, (FIG. 12E) colchicine and (FIG. 12F) LLOMe. Scale bar in (FIG. 12F) represents 10 μm and applies to all images.

FIGS. 13A-13C show theoretical analysis of phagosomal rupture caused by PNIPAM microparticles. Model-calculated osmotic pressure or osmotic-pressure difference vs. radius of the microparticle/phagosome at (FIG. 13A) 6.4° C., (FIG. 13B) 11° C. and (FIG. 13C) 22° C.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

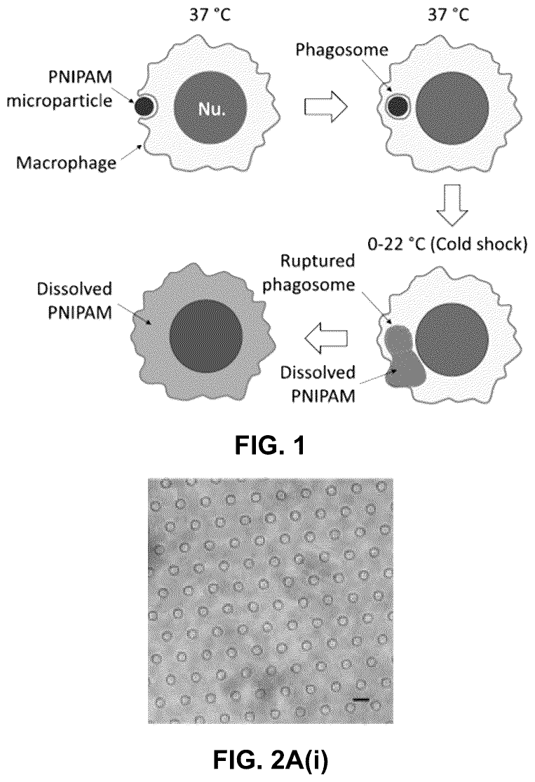

Disclosed herein are microfabricated microparticles composed of uncrosslinked linear ploy(N-isopropylacrylamide)(PNIPAM) as phagocytic objects. In one aspect, PNIPAM is a synthetic polymer that can undergo phase transition in water at a lower critical solution temperature (LCST) of around 32° C. In another aspect, PNIPAM chains are typically soluble in water at temperatures well below LCST, but they tend to aggregate and form an insoluble macroscopic gel phase at temperatures above LCST, such as 37° C. The disclosed method, shown schematically in FIG. 1, starts with the phagocytosis of a PNIPAM microparticle by a macrophage at 37° C. Subsequently, the macrophage is briefly exposed to a temperature between 0 and 22° C., a process called cold shock, which renders the PNIPAM dissolved in water. The dissolved PNIPAM generates a high osmotic pressure inside the phagosome, leading to its rupture and the release of the dissolved PNIPAM into the cytoplasm of the macrophage.

In an aspect, the disclosed method offers several advantages over existing techniques for inducing phagosomal rupture in macrophages. In a further aspect, the method relies solely on osmotic pressure to induce phagosomal rupture. In another aspect, the disclosed method utilizes uncrosslinked linear PNIPAM to generate osmotic pressure. In a still further aspect, the disclosed method utilizes a microfabrication technique to produce the PNIPAM microparticles with monodispersed geometry and composition.

Disclosed herein is a method for producing loaded polymeric microparticles in one or more phagocytic cells, the method including at least the steps of:
(a) medium with the one or more phagocytic cells, wherein, during incubation, the loaded polymeric microparticles are taken up by the phagocytic cells into phagosomes, wherein the loaded polymeric microparticles include one or more nanoparticles;
(b) transferring the phagocytic cells containing loaded polymeric microparticles to a second medium at a second temperature at which the loaded polymeric microparticles swell;
(c) incubating the phagocytic cells at the second temperature, wherein during incubation, the phagosomes rupture in the phagocytic cells; and
(d) transferring the phagocytic cells to a third medium at a third temperature, to produce the loaded microparticles in the phagocytic cells.

In another aspect, the method includes the step of loading polymeric microparticles with one or more nanoparticles to form the loaded polymeric microparticles prior to step (a). FIG. 1 includes a non-limiting example of the disclosed method. In one aspect, the loaded polymeric microparticles can include or be made from polymeric microparticles poly(N-isopropylacrylamide) (PNIPAM), a copolymer thereof, a derivative thereof, or any combination thereof. In a further aspect, the copolymer of PNIPAM can be PNIPAM-fluorescein. In one aspect, the PNIPAM has a molecular weight of from about 1000 Da to about 300,000 Da, or of about 1000, 5000, 10,000, 20,000, 30,000, 40,000, 50,000, 75,000, 100,000, 125,000, 150,000, 175,000, 200,000, 225,000, 250,000, 275,000, or about 300,000 Da, or a combination of any of the foregoing values, or a range encompassing any of the foregoing values.

In one aspect, PNIPAM-based copolymers and/or mixtures of PNIPAM or derivatives thereof with other polymers can be used in the disclosed methods.

In another aspect, the loaded polymeric microparticles have an average particle of from about 0.1 μm to about 20 μm, or about 0.1, 0.5, 1, 2, 3, 4, 5, 6, 7, 8, 9, 10, 11, 12, 13, 14, 15, 16, 17, 18, 19, or about 20 μm, or a combination of any of the foregoing values, or a range encompassing any of the foregoing values.

In some aspects, the one or more nanoparticles can include an anti-cancer agent, a fluorescent molecule, a metal or metal oxide, a live microorganism, an inactivated microorganism, a component of an inactivated microorganism, a polysaccharide or derivative thereof, DNA, or any combination thereof. In one aspect, the metal oxide can be iron oxide. In another aspect, the polysaccharide or derivative thereof can be zymosan, lipopolysaccharide, or any combination thereof. In another aspect, the one or more nanoparticles have an average particle diameter of from about 5 nm to about 1 μm, or of about 5, 25, 50, 100, 150, 200, 250, 300, 350, 400, 450, 500, 550, 600, 650, 700, 750, 800, 850, 900, 950, or about 1000 nm (1 μm), or a combination of any of the foregoing values, or a range encompassing any of the foregoing values. In one aspect, the one or more nanoparticles or components thereof can polarize a macrophage to an inflammatory phenotype. In another aspect, the nanoparticles can include components of inactivated microorganisms, wherein the microorganisms can be selected from bacteria, fungi, and viruses.

In one aspect, the first temperature is from about 33° C. to about 40° C., or is about 33, 34, 35, 36, 37, 38, 39, or about 40° C., or a combination of any of the foregoing values, or a range encompassing any of the foregoing values. In another aspect, the second temperature is from about 0° C. to about 22° C., or is about 0, 1, 2, 3, 4, 5, 6, 7, 8, 9, 10, 11, 12, 13, 14, 15, 16, 17, 18, 19, 20, 21, or about 22° C., or a combination of any of the foregoing values, or a range encompassing any of the foregoing values. In still another aspect, the third temperature is from about 33° C. to about 40° C., or is about 33, 34, 35, 36, 37, 38, 39, or about 40° C., or a combination of any of the foregoing values, or a range encompassing any of the foregoing values. In another aspect, the first medium can be a cell culture medium, the second medium can be a cell culture medium, phosphate buffered saline (PBS), or any combination thereof, and the third medium can be a cell culture medium. In one aspect, and without wishing to be bound by theory, the first temperature should be above the glass transition temperature of the polymeric material in the microparticles, but should not be high enough to kill or severely injure the phagocytic cells. In one aspect, the cell culture medium of the first medium and, optionally, the second medium when PBS is not used, can be Dulbecco's Modified Eagle's Medium (DMEM) with 4.5 g/L glucose and 4 mM L-glutamine, supplemented with 10% fetal bovine serum, 100 units/mL of penicillin and 100 μg/mL streptomycin. In one aspect, when the second medium is PBS, the PBS can have a 1× concentration. In another aspect, the PBS can be 0.6×. In a further aspect, and without wishing to be bound by theory, 0.6×PBS may enhance phagosome rupture.

In any of these aspects, the microparticles are non-toxic and are not biodegradable.

Also disclosed herein are cytosolic microparticles made by the disclosed methods.

In one aspect, disclosed herein is a phagocytic cell including a polymeric microparticle loaded with one or more nanoparticles. Also disclosed herein are phagocytic cells including loaded polymeric microparticles in the cytosol of the phagocytic cells. In another aspect, the polymeric microparticle can include or be made from poly(N-isopropylacrylamide) (PNIPAM),), a copolymer thereof, a derivative thereof, or any combination thereof. In one aspect, the copolymer of PNIPAM can be PNIPAM-fluorescein. In one aspect, the PNIPAM, copolymer thereof, or derivative thereof has a molecular weight of from about 1000 Da to about 300,000 Da, or of about 1000, 5000, 10,000, 20,000, 30,000, 40,000, 50,000, 75,000, 100,000, 125,000, 150,000, 175,000, 200,000, 225,000, 250,000, 275,000, or about 300,000 Da, or a combination of any of the foregoing values, or a range encompassing any of the foregoing values.

In another aspect, the polymeric microparticle can have an average particle of from about 0.1 μm to about 20 μm, or about 0.1, 0.5, 1, 2, 3, 4, 5, 6, 7, 8, 9, 10, 11, 12, 13, 14, 15, 16, 17, 18, 19, or about 20 μm, or a combination of any of the foregoing values, or a range encompassing any of the foregoing values. In some aspects, the one or more nanoparticles can include an anti-cancer agent, a fluorescent molecule, a metal or metal oxide, a live microorganism, an inactivated microorganism, a component of an inactivated microorganism, a polysaccharide or derivative thereof, DNA, or any combination thereof. In one aspect, the metal oxide can be iron oxide. In another aspect, the polysaccharide or derivative thereof can be zymosan, lipopolysaccharide, or any combination thereof. In another aspect, the one or more nanoparticles have an average particle diameter of from about 5 nm to about 1 μm, or of about 5, 25, 50, 100, 150, 200, 250, 300, 350, 400, 450, 500, 550, 600, 650, 700, 750, 800, 850, 900, 950, or about 1000 nm (1 μm), or a combination of any of the foregoing values, or a range encompassing any of the foregoing values. In any of these aspects, the cytosolic microparticles are non-toxic and are not biodegradable.

In a still further aspect, disclosed herein are phagocytic cells including the disclosed cytosolic microparticles. In one aspect, the phagocytic cells can be macrophages, dendritic cells, neutrophils, monocytes, mast cells, or non-professional phagocytic cells such as, for example, epithelial cells and/or fibroblasts.

Also disclosed herein are compositions including the disclosed phagocytic cells. In some aspects, the compositions further include at least one excipient. In a further aspect, the excipient can include saline.

In another aspect, disclosed herein is a method for treating a disease, the method including administering the disclosed phagocytic cells and/or compositions to a subject. In one aspect, the phagocytic cells and/or compositions are delivered to the subject intravenously. In one aspect, the disease is cancer, rheumatoid arthritis, atherosclerosis, Alzheimer's disease, multiple sclerosis, obesity, or another disease characterized by chronic local inflammation. In one aspect, the disease can be any disease that involves accumulation of macrophages derived from circulating macrophages or monocytes. In one aspect, when the disease is cancer, the disclosed methods can polarize macrophages into cancer-fighting phenotypes using the nanoparticles, microparticles, and compositions. In another aspect, for a disease characterized by inflammation, the disclosed methods can polarize macrophages to an anti-inflammation phenotype with the disclosed nanoparticles, microparticles, and compositions. In another aspect, the cancer can be non-Hodgkins lymphoma, neuroblastoma, sarcoma, metastatic brain cancers, ovarian cancer, prostate cancer, breast cancer, lymphoma, non-small cell lung carcinoma, gastric cancer, gastroesophageal junction adenocarcinoma, melanoma, squamous cell carcinoma, pancreatic cancer, hepatocellular carcinoma, colorectal cancer, angiosarcoma, head and neck cancer, ovarian cancer, solid tumors, multiple myeloma, glioblastoma, testicular cancer, urothelial cancer, adenocortical carcinoma, clear cell renal cell carcinoma, small cell lung renal cell carcinoma, nasopharyngeal cancer, glioma, gall bladder cancer, thyroid tumor, bone cancer, cervical cancer, uterine cancer, endometrial cancer, vulvar cancer, bladder cancer, colon cancer, colorectal cancer, pancreatic cancer, neuronal cancers, mesothelioma, cholangiocarcinoma, small bowel adenocarcinoma, epidermoid carcinoma, cancer of the pleural or peritoneal membranes, another cancer, or any combination thereof.

In one aspect, the method can be performed once, twice, or more times during the duration of disease treatment. In a further aspect, when the method is performed more than once, the method can be performed at intervals of from about 1 month to about 6 months.

In some aspects, the disclosed method further includes administering an additional treatment to the subject, including, but not limited to, radiation, chemotherapy, immunotherapy, bone marrow transplant, hormone therapy, surgery, or any combination thereof.

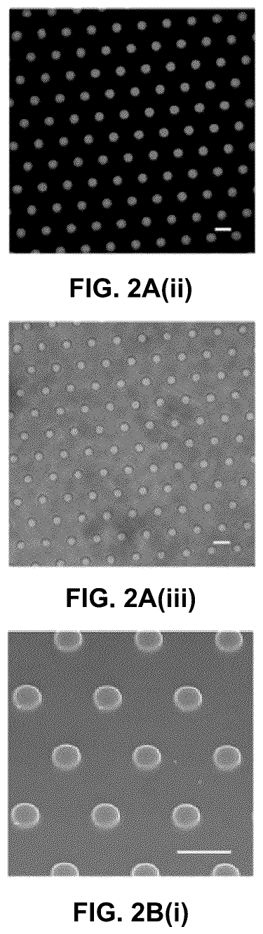

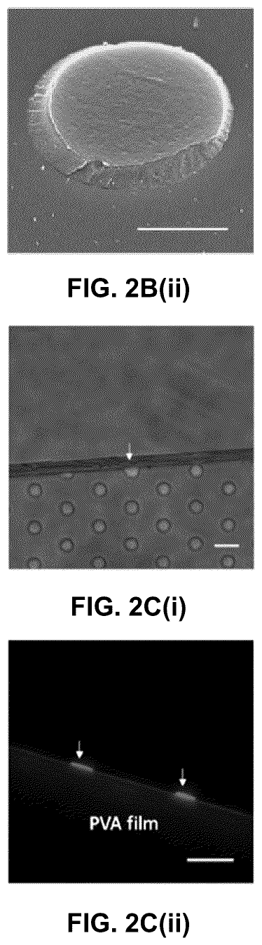

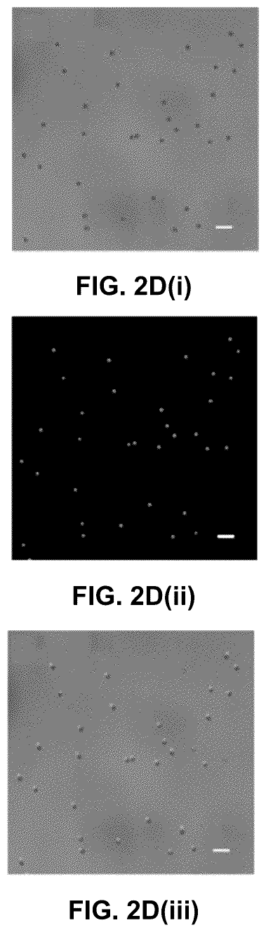

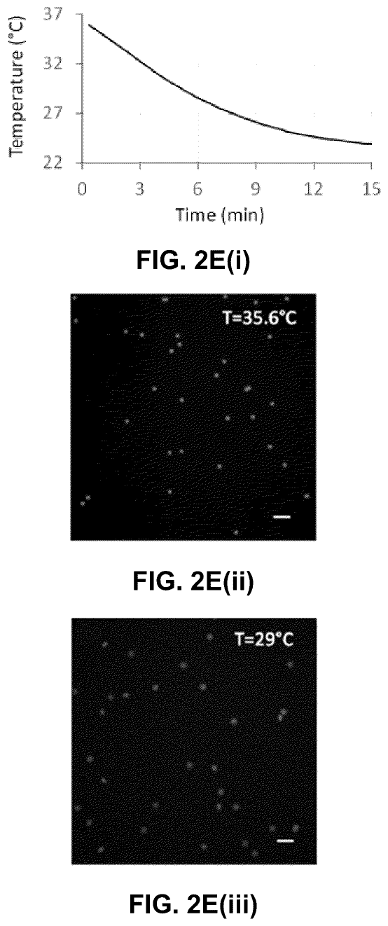

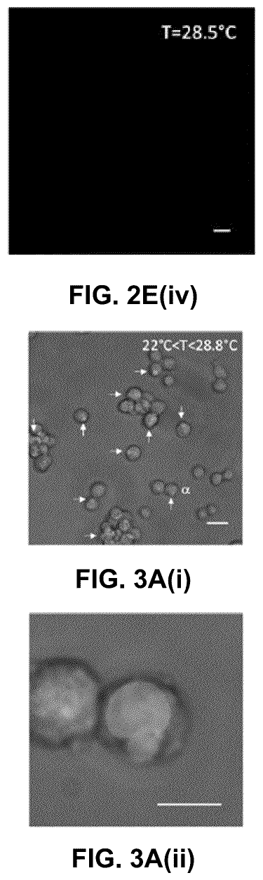

In one aspect, the phagocytic cells are isolated from the subject prior to incubating the phagocytic cells with the loaded polymeric microparticles (see FIG. 2).

In one aspect, the subject can be a mammal or a bird. In one aspect, the mammal can be a human, dog, cat, hamster, rabbit, guinea pig, mouse, rat, sheep, goat, cow, horse, or pig. In another aspect, the bird can be a turkey, duck, chicken, goose, or parrot.

Also disclosed herein are kits including:
(a) loaded polymeric microparticles, wherein the loaded polymeric microparticles comprise one or more nanoparticles; and
(b) instructions for introducing the loaded polymeric microparticles into phagocytic cells.

In one aspect, the kit can further include at least one cell culture medium, phosphate buffered saline (PBS), or any combination thereof. In a further aspect, the loaded polymeric microparticles can include or be made from poly(N-isopropylacrylamide) (PNIPAM), a copolymer thereof, a derivative thereof, or any combination thereof. In one aspect, the copolymer of PNIPAM can be PNIPAM-fluorescein. In still another aspect, the PNIPAM can have a molecular weight of from about 1000 Da to about 300,000 Da, or of about 1000, 5000, 10,000, 20,000, 30,000, 40,000, 50,000, 75,000, 100,000, 125,000, 150,000, 175,000, 200,000, 225,000, 250,000, 275,000, or about 300,000 Da, or a combination of any of the foregoing values, or a range encompassing any of the foregoing values. In one aspect, the loaded polymeric microparticles can have an average particle diameter of from about 0.1 μm to about 20 μm, or about 0.1, 0.5, 1, 2, 3, 4, 5, 6, 7, 8, 9, 10, 11, 12, 13, 14, 15, 16, 17, 18, 19, or about 20 μm, or a combination of any of the foregoing values, or a range encompassing any of the foregoing values.

In one aspect, the one or more nanoparticles can include an anti-cancer agent, a fluorescent molecule, a metal or metal oxide, a live microorganism, an inactivated microorganism, a component of an inactivated microorganism, a polysaccharide or derivative thereof, DNA, or any combination thereof. In a further aspect, the metal oxide can be an iron oxide. In another aspect, the polysaccharide or derivative thereof can be zymosan, lipopolysaccharide, or any combination thereof. In another aspect, the one or more nanoparticles can have an average particle diameter of from about 5 nm to about 1 μm, or of about 5, 25, 50, 100, 150, 200, 250, 300, 350, 400, 450, 500, 550, 600, 650, 700, 750, 800, 850, 900, 950, or about 1000 nm (1 μm), or a combination of any of the foregoing values, or a range encompassing any of the foregoing values.

In one aspect, the disclosed method allows delivery of PNIPAM microparticles containing nanoparticles into cytosol of phagocytic cells, meaning that the microparticles are not located in phagosomes. As a result, the nanoparticles are not exposed to an acidic and degradative environment as in typical mature phagosomes. In a further aspect, material transport between the nanoparticles and cytosol is not inhibited by the lipid membrane as phagosomes. In still another aspect, the nanoparticles are embedded in the interior of the microparticles. Consequently, they do not physically contact organelles in the cells. Further in this aspect, lack of contact between nanoparticles and organelles eliminates any risk of nanoparticle interference with normal functions of organelles. In a still further aspect, the nanoparticles are not subject to mechanisms such as exocytosis.

In one aspect, the PNIPAM matrix of the microparticles is hydrated. As a result, molecules and ions can diffuse easily within the microparticles. In still another aspect, a wide variety of nanoparticles with therapeutic functions can be loaded into the PNIPAM microparticles. In any of these aspects, the disclosed method does not require the genetic modification of the macrophages or other phagocytic cells.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a phagocytic cell,” “a nanoparticle,” or “an anti-cancer agent,” include, but are not limited to, mixtures or combinations of two or more such phagocytic cells, nanoparticles, or anti-cancer agents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less' and should be interpreted to include the specific ranges of ‘about x,’ ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a nanoparticle refers to an amount that is sufficient to achieve the desired improvement in the property modulated by the formulation component, e.g. achieving or retaining an anti-cancer phenotype in a macrophage. The specific level in terms of wt % in a composition required as an effective amount will depend upon a variety of factors including the type and size of tumor being treated, chemical components of the nanoparticle, number of microparticle/nanoparticle treatments administered to a subject, additional (i.e., non-nanoparticle) treatments administered to the subject, and the like.

As used herein, the terms “optional” or “optionally” means that the subsequently described event or circumstance can or cannot occur, and that the description includes instances where said event or circumstance occurs and instances where it does not.

“Phagocytic cells” or “phagocytes” as used herein refers to any cell that can perform the process of phagocytosis. In mammals and other vertebrates, for example, phagocytic cells include numerous white blood cells such as, for example, macrophages, neutrophils, monocytes, mast cells, dendritic cells, and the like.

A “phagosome” as used herein refers to a vesicle in the interior of a phagocytic cell, wherein the vesicle surrounds a particle taken in by the process of phagocytosis. In one aspect, in the disclosed methods, the phagosomes are disrupted without harming other cellular membranes, thus allowing the disclosed microparticles to enter the cytosol.

“Cytosol” or “cytosolic” as used herein refers to the aqueous component of the cytoplasm of a cell. Organelles (both membrane-bound and not membrane-bound) and other particles are suspended in the cytosol. In one aspect, the polymeric microparticles disclosed herein are present in the cytosol of the disclosed phagocytic cells (i.e., are not separated from the cytosol by any membrane).

As used herein, “excipient” refers to an inactive ingredient in a medication. In one aspect, excipients can include stabilizers, carriers, solvents, buffers, coloring agents, fillers, viscosity modifiers, solubility enhancers, and the like. Exemplary excipients are described below.

Unless otherwise specified, pressures referred to herein are based on atmospheric pressure (i.e. one atmosphere).

### Excipients and Carriers

The phagocytic cells and compositions described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for intravenous injection or parenteral administration. Carriers include solids or liquids, and the type of carrier is chosen based on the type of administration being used.

The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered Aspects, which should not be confused with the claims.

Aspect 1. A method for introducing loaded polymeric microparticles into one or more phagocytic cells, the method comprising:
(a) incubating loaded polymeric microparticles at a first temperature and in a first medium with the one or more phagocytic cells, wherein, during incubation, the loaded polymeric microparticles are taken up by the phagocytic cells into phagosomes, wherein the loaded polymeric microparticles comprise one or more nanoparticles;
(b) transferring the phagocytic cells containing loaded polymeric microparticles to a second medium at a second temperature at which the loaded polymeric microparticles swell;
(c) incubating the phagocytic cells at the second temperature, wherein during incubation, the phagosomes rupture in the phagocytic cells; and
(d) transferring the phagocytic cells to a third medium at a third temperature, to produce the loaded microparticles in the phagocytic cells.

Aspect 2. The method of aspect 1, further comprising loading polymeric microparticles with one or more nanoparticles to form the loaded polymeric microparticles prior to step (a).

Aspect 3. The method of aspect 1 or 2, wherein the loaded polymeric microparticles comprise poly(N-isopropylacrylamide) (PNIPAM), a copolymer thereof, a derivative thereof, or any combination thereof.

Aspect 4. The method of aspect 3, wherein the copolymer of PNIPAM comprises PNIPAM-fluorescein.

Aspect 5. The method of aspect 3 or 4, wherein the PNIPAM, copolymer thereof, or derivative thereof, has a molecular weight of from about 1000 Da to about 300,000 Da.

Aspect 6. The method of aspect 5, wherein the PNIPAM, copolymer thereof, or derivative thereof has a molecular weight of about 40,000 Da.

Aspect 7. The method of any one of aspects 1-6, wherein the loaded polymeric microparticles have an average particle diameter of from about 0.1 μm to about 20 μm.

Aspect 8. The method of any one of aspects 1-7, wherein the one or more nanoparticles comprise an anti-cancer agent, a fluorescent molecule, a metal or metal oxide, a live microorganism, an inactivated microorganism, a component of an inactivated microorganism, a polysaccharide or derivative thereof, DNA, or any combination thereof.

Aspect 9. The method of aspect 8, wherein the metal oxide comprises iron oxide.

Aspect 10. The method of aspect 8, wherein the polysaccharide or derivative thereof comprises zymosan, lipopolysaccharide, or any combination thereof.

Aspect 11. The method of any one of aspects 1-10, wherein the one or more nanoparticles have an average particle diameter of from about 5 nm to about 1 μm.

Aspect 12. The method of aspect 11, wherein the one or more nanoparticles have an average particle diameter of about 100 nm.

Aspect 13. The method of any one of aspects 1-12, wherein the first temperature is from about 33° C. to about 40° C.

Aspect 14. The method of aspect 13, wherein the first temperature is about 37° C.

Aspect 15. The method of any one of aspects 1-14, wherein the first medium comprises a cell culture medium.

Aspect 16. The medium of any one of aspects 1-15, wherein the second temperature is from about 0° C. to about 22° C.

Aspect 17. The method of aspect 16, wherein the second temperature is about 0° C.

Aspect 18. The method of any one of aspects 1-17, wherein the second medium comprises a cell culture medium, phosphate buffered saline (PBS), or any combination thereof.

Aspect 19. The method of any one of aspects 18, wherein the third temperature is from about 33° C. to about 40° C.

Aspect 20. The method of aspect 19, wherein the third temperature is about 37° C.

Aspect 21. The method of any one of aspects 1-20, wherein the third medium comprises a cell culture medium.

Aspect 22. The method of any one of aspects 1-21, wherein the loaded polymeric microparticles are non-toxic.

Aspect 23. The method of any one of aspects 1-22, wherein the loaded polymeric microparticles are not biodegradable.

Aspect 24. A phagocytic cell comprising loaded polymeric microparticles made by the method of any one of aspects 1-23.

Aspect 25. A phagocytic cell comprising loaded polymeric microparticles in the cytosol of the phagocytic cell.

Aspect 26. The phagocytic cell of aspect 25, wherein the loaded polymeric microparticles comprise poly(N-isopropylacrylamide) (PNIPAM), a copolymer thereof, a derivative thereof, or any combination thereof.

Aspect 27. The phagocytic cell of aspect 26, wherein the copolymer of PNIPAM comprises PNIPAM-fluorescein.

Aspect 28. The phagocytic cell of any one of aspects 25-27 wherein the PNIPAM has a molecular weight of from about 1000 Da to about 300,000 Da.

Aspect 29. The phagocytic cell of aspect 28, wherein the PNIPAM has a molecular weight of about 40,000 Da.

Aspect 30. The phagocytic cell of any one of aspects 24-29, wherein the loaded polymeric microparticles have an average particle diameter of from about 0.1 μm to about 20 μm.

Aspect 31. The phagocytic cell of any one of aspects 24-30, wherein the one or more nanoparticles comprise an anti-cancer agent, a fluorescent molecule, a metal or metal oxide, a live microorganism, an inactivated microorganism, a component of an inactivated microorganism, a polysaccharide or derivative thereof, DNA, or any combination thereof.

Aspect 32. The phagocytic cell of aspect 31, wherein the metal oxide comprises iron oxide.

Aspect 33. The phagocytic cell of aspect 31, wherein the polysaccharide or derivative thereof comprises zymosan, lipopolysaccharide, or any combination thereof.

Aspect 34. The phagocytic cell of any one of aspects 24-33, wherein the one or more nanoparticles have an average particle diameter of from about 5 nm to about 1 μm.

Aspect 35. The phagocytic cell of aspect 34, wherein the one or more nanoparticles have an average particle diameter of about 100 nm.

Aspect 36. The phagocytic cell of any one of aspects 24-35, wherein the loaded polymeric microparticle is non-toxic.

Aspect 37. The phagocytic cell of any one of aspects 24-36, wherein the loaded polymeric microparticle is not biodegradable.

Aspect 38. The phagocytic cell of any one of aspects 24-37, wherein the phagocytic cell comprises a macrophage, a dendric cell, a neutrophil, a monocytes, a mast cell, or a non-professional phagocytic cell.

Aspect 39. The phagocytic cell of aspect 38, wherein the non-professional phagocytic cell comprises an epithelial cell or a fibroblast.

Aspect 40. A composition comprising the phagocytic cell of any one of aspects 24-39.

Aspect 41. The composition of aspect 40, further comprising at least one excipient.

Aspect 42. The composition of aspect 41, wherein the at least one excipient comprises saline.

Aspect 43. A method for treating a disease in a subject, the method comprising administering the phagocytic cell according to any one of aspects 24-39 or the composition of any one of aspects 40-42 to the subject.

Aspect 44. The method of aspect 43, wherein the phagocytic cell or the composition is administered to the subject intravenously.

Aspect 45. The method of aspect 43 or 44, wherein the disease comprises cancer, rheumatoid arthritis, atherosclerosis, Alzheimer's disease, multiple sclerosis, obesity, or another disease characterized by chronic local inflammation.

Aspect 46. The method of aspect 45, wherein the cancer comprises non-Hodgkins lymphoma, neuroblastoma, sarcoma, metastatic brain cancers, ovarian cancer, prostate cancer, breast cancer, lymphoma, non-small cell lung carcinoma, gastric cancer, gastroesophageal junction adenocarcinoma, melanoma, squamous cell carcinoma, pancreatic cancer, hepatocellular carcinoma, colorectal cancer, angiosarcoma, head and neck cancer, ovarian cancer, solid tumors, multiple myeloma, glioblastoma, testicular cancer, urothelial cancer, adenocortical carcinoma, clear cell renal cell carcinoma, small cell lung renal cell carcinoma, nasopharyngeal cancer, glioma, gall bladder cancer, thyroid tumor, bone cancer, cervical cancer, uterine cancer, endometrial cancer, vulvar cancer, bladder cancer, colon cancer, colorectal cancer, pancreatic cancer, neuronal cancers, mesothelioma, cholangiocarcinoma, small bowel adenocarcinoma, epidermoid carcinoma, cancer of the pleural or peritoneal membranes, another cancer, or any combination thereof.

Aspect 47. The method of any one of aspects 43-46, wherein the method is performed once.

Aspect 48. The method of any one of aspects 43-46, wherein the method is performed two or more times.

Aspect 49. The method of aspect 48, wherein the method is performed at intervals of from about 1 to about 6 months.

Aspect 50. The method of any one of aspects 43-49, further comprising administering an additional treatment to the subject.

Aspect 51. The method of aspect 50, wherein the additional treatment comprises radiation, chemotherapy, immunotherapy, bone marrow transplant, hormone therapy, surgery, or any combination thereof.

Aspect 52. The method of any one of aspects 43-51, further comprising isolating the phagocytic cells from the subject prior to incubating the phagocytic cells with the loaded polymeric microparticles.

Aspect 53. The method of any one of aspects 43-52, wherein the subject is a mammal or a bird.

Aspect 54. The method of aspect 53, wherein the mammal is a human, dog, cat, hamster, rabbit, guinea pig, mouse, rat, sheep, goat, cow, horse, or pig.

Aspect 55. The method of aspect 53, wherein the bird is a turkey, duck, chicken, goose, or parrot.

Aspect 56. A kit comprising:
(a) loaded polymeric microparticles, wherein the loaded polymeric microparticles comprise one or more nanoparticles; and
(b) instructions for introducing the loaded polymeric microparticles into phagocytic cells.

Aspect 57. The kit of aspect 56, further comprising at least one cell culture medium, phosphate buffered saline, or any combination thereof.

Aspect 58. The kit of aspect 56 or 57, wherein the loaded polymeric microparticles comprise poly(N-isopropylacrylamide) (PNIPAM), a copolymer thereof, a derivative thereof, or any combination thereof.

Aspect 59. The kit of aspect 58, wherein the copolymer of PNIPAM comprises PNIPAM-fluorescein.

Aspect 60. The kit of any one of aspects 56-58, wherein the PNIPAM, copolymer thereof, or derivative thereof has a molecular weight of from about 1000 Da to about 300,000 Da.

Aspect 61. The kit of aspect 60, wherein the PNIPAM, copolymer thereof, or derivative thereof has a molecular weight of about 40,000 Da.

Aspect 62. The kit of any one of aspects 56-59, wherein the loaded polymeric microparticles have an average particle diameter of from about 0.1 μm to about 20 μm.

Aspect 63. The kit of any one of aspects 56-60, wherein the one or more nanoparticles comprise an anti-cancer agent, a fluorescent molecule, a metal or metal oxide, a live microorganism, an inactivated microorganism, a component of an inactivated microorganism, a polysaccharide or derivative thereof, DNA, or any combination thereof.

Aspect 64. The kit of aspect 63, wherein the metal oxide comprises iron oxide.

Aspect 65. The kit of aspect 63, wherein the polysaccharide or derivative thereof comprises zymosan, lipopolysaccharide, or any combination thereof.

Aspect 66. The kit of any one of aspects 56-65, wherein the one or more nanoparticles have an average particle diameter of from about 5 nm to about 1 μm.

Aspect 67. The kit of aspect 66, wherein the one or more nanoparticles have an average particle diameter of about 100 nm.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

### Materials

Poly(N-isopropylacrylamide) (PNIPAM) with a molecular weight (MW) of ˜40 kDa was purchased from Polysciences (product number: 21458). A 2.5% solid-content aqueous suspension of red fluorescent sulfate-modified polystyrene nanoparticles with a mean diameter of 100 nm (product number: L9902) and lipopolysaccharides (LPSs) from Escherichia coli O111::B4 (product number: L2630) were purchased from Sigma-Aldrich. Poly(vinyl alcohol) (PVA, 88% hydrolyzed, MW=3 kDa) was purchased from Scientific Polymer Products (catalog number: 336). The poly(dimethyl siloxane) (PDMS) kit (Sylgard 184) was purchased from Dow Corning. RAW264.7 macrophages were purchased from the American Type Culture Collection. Dulbecco's modified Eagle's medium (DMEM, with 4.5 g/L glucose and 4 mM L-glutamine) was purchased from VWR. Fetal bovine serum (FBS) was a product of Avantor and purchased from VWR. Hoechst 33342 was purchased from Life Technologies. LysoView 488 (LysoView 488) was purchased from Biotium. Chloroquine was a product of Chem-Impex International and purchased from VWR. Tetrandrine was a product of TCI America and purchased from VWR. Colchicine was a product of Adipogen Corporation and purchased from VWR. L-Leucyl-L-leucine methyl ester hydrochloride (LLOMe) was purchased from Santa Cruz Biotechnology. Acetone, phosphate-buffered saline (PBS), dimethyl sulfoxide (DMSO), and glass coverslips were purchased from VWR.

### Synthesis and Characterization of Poly(N-Isopropylacrylamide-Co-Fluorescein-O-Acrylate)

Poly(N-isopropylacrylamide-co-fluorescein-O-acrylate) (PNIPAM-fluorescein hereafter) was synthesized and characterized as described previously.

### Cell Culture

RAW264.7 macrophages were maintained in a complete medium (DMEM supplemented with 10% FBS, 100 units/mL penicillin, and 100 μg/mL streptomycin) at 37° C. with 5% CO₂ in a standard incubator.

### Preparation of PDMS Stamps

PDMS stamps were prepared as previously described. The stamps carried 5 μm-radius circular pillars in a hexagonal lattice with a pillar-to-pillar distance of 30 μm. The height of the pillars was ˜6 μm.

### Preparation of PVA-Coated Glass Coverslips

An aqueous solution of PVA (5 wt %, 100 μL) was deposited onto a glass coverslip to cover a circular area with a diameter of ˜1.5 cm and was allowed to dry in air at 22° C.

### Fabrication of Microparticles

Two types of microparticles were created, characterized, and employed. The first type was composed of commercial PNIPAM and labeled with fluorescent nanoparticles. These microparticles were produced on either a bare glass coverslip or a PVA-coated glass coverslip. The second type of microparticles consisted solely of PNIPAM-fluorescein and were produced on a PVA-coated glass coverslip.

PNIPAM microparticles were printed on a bare glass coverslip. The procedure consists of the following three steps. (1) An aqueous solution of PNIPAM (30 wt %), the aqueous suspension of fluorescent nanoparticles, and distilled water were mixed at a volume ratio of 12:2:1. (2) The mixture (30 μL) was spread on a stamp mounted on a spin coater, and the stamp was spun at 3000 revolutions per minute (rpm) for 90 s (3) The stamp was placed on a glass coverslip on a hotplate set at ˜97° C. with a mild compression applied manually for ˜26 s before being peeled off. This step is referred to as printing.

PNIPAM microparticles were printed on a PVA-coated glass coverslip. The procedure consists of three steps. The first two steps are the same as those for fabricating PNIPAM microparticles on a glass coverslip. In the third step, the stamp was gently placed on a PVA-coated glass coverslip on a hotplate set at 93° C. and kept for ˜10 s before being peeled off.

PNIPAM-fluorescein microparticles were printed on a PVA-coated glass coverslip. The procedure consists of the following three steps. (1) A solution of PNIPAM-fluorescein in acetone (15 wt %) was prepared. (2) The solution (60 μL) was spread on a stamp mounted on a spin coater, and the stamp was spun at 3000 rpm for 60 s (3) The stamp was placed on a PVA-coated glass coverslip on a hotplate set at 93° C. with a mild compression applied manually for ˜10 s before being peeled off. The PVA film that carried the microparticles could be detached from the coverslip as a free-standing microparticle-carrying PVA film when necessary.

### Characterization of Microparticles

FIGS. 2A(i)-2A(iii). The PNIPAM microparticles were printed on a glass coverslip and imaged with an optical microscope.

FIGS. 2B(i)-2B(ii). The PNIPAM microparticles were printed on a glass coverslip. The sample was sputter-coated with a 10 nm-thick layer of gold and imaged with a FEI Helios G4 UC dual-beam (electron and Ga ion) field emission scanning electron microscope (SEM) under low-vacuum conditions.

FIGS. 2C(i)-2C(ii). The PNIPAM microparticles were printed on a PVA-coated glass coverslip. The PVA film carrying the microparticles was delaminated from the coverslip as a free-standing film with a razor blade. The PVA film is cut into two pieces with a razor blade at the area containing the microparticles. The cutting edge of a piece of the PVA film was imaged when the film was lying flat to obtain FIG. 2C(i). The cross section of the cutting edge was imaged to obtain FIG. 2C(ii).

FIGS. 2D(i) and 2D(iii). A PDMS film (thickness=7 mm) with a circular through hole (diameter=19 mm) was placed on a PNIPAM microparticle-carrying PVA-coated glass coverslip with the micro-particles being enclosed by the hole. The space created by the hole and the underlying coverslip will be referred to as a chamber hereafter. The assembled structure, named as device hereafter, was prewarmed in an oven (37° C.). The prewarmed PBS (37° C., 1 mL) was added into the chamber, followed by placing a 3 mm-thick PDMS film atop to cover the hole. The whole structure was then placed in a plastic Petri dish, and the dish was kept in an oven set at 37° C. for 7 d. The PNIPAM microparticles were imaged immediately (within 2 min) after the device was taken out of the oven.

FIGS. 2E(i)-2E(iv). The setup used to obtain FIG. 2D(i)-2D(iii) was used with the prewarmed complete medium (37° C.). After the complete medium was added into the chamber, the device was kept in an incubator at 37° C. for 3 h. After the incubation, the device was taken out of the incubator and placed on an optical microscope for imaging. The temperature of the complete medium in the chamber of the device was measured with a Fluke 179 True-rms Digital Multimeter equipped with a thermocouple probe. Simultaneously, the microparticles were imaged using the microscope until they disappeared due to dissolution.

### Characterization of Phagosomal Rupture

The same type of devices as those used for characterizing the microparticles were used. Each device was sterilized by exposing it to UV light in a laminar hood for 20 min before use.

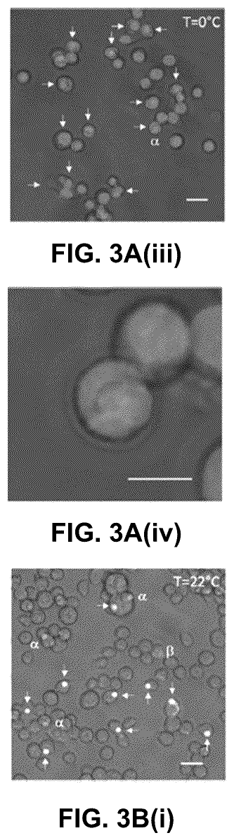

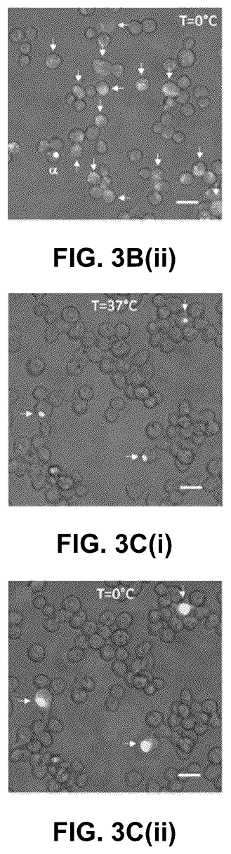

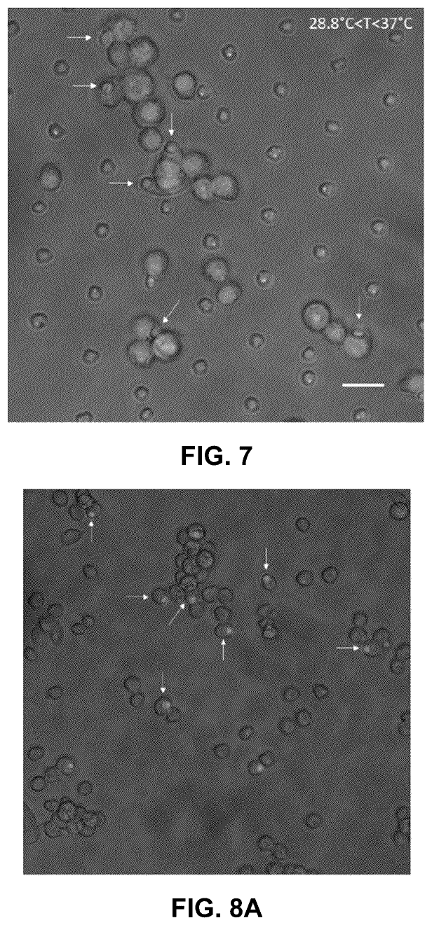

FIGS. 3A(i)-3A(iv) and 7. A prewarmed suspension of macro-phages in the complete medium (3.5×10⁵ cells/mL, 1 mL, 37° C.) supplemented with LPS (1 μg/mL) was added onto an array of PNIPAM microparticles on a bare glass coverslip in a device. The macrophages were incubated (5% CO₂, 37° C.). After 3 h, Hoechst 33342 (1 μg/mL) was added into the culture and incubated (5% CO₂, 37° C.) for 30 min. The culture was imaged in the complete medium immediately after it was taken out of the incubator to obtain FIG. 7. FIG. 3A(i) was obtained as several minutes elapsed. To obtain FIG. 3A(iii), the complete medium in a culture was replaced with 0° C. PBS immediately (within 30 s) after the culture was taken out of the incubator. The culture was then placed on ice and maintained for 2 min before being imaged.

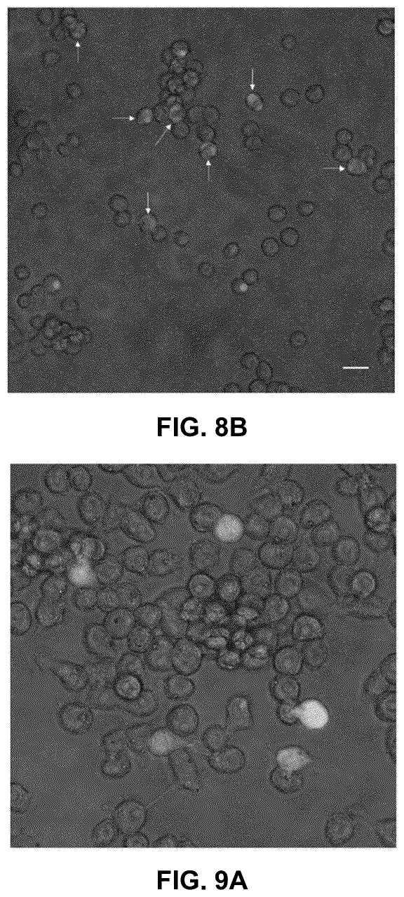

FIGS. 8A-8B. A prewarmed suspension of macrophages in the complete medium (3.5×10⁵ cells/mL, 1 mL, 37° C.) supplemented with LPS (1 μg/mL) was added onto an array of PNIPAM microparticles on a bare glass coverslip in a device. The macrophages were incubated (5% CO₂, 37° C.). After 3 h, the culture was mounted on the stage of the microscope. An area of interest was identified. An image of the area was recorded after the microparticles that were not colocalized with macrophages had disappeared as FIG. 8A. The complete medium of the culture was then replaced with 0° C. PBS, followed by recording a series of fluorescent nanoparticle images of the area at a rate of one image per second. Next, an image of the area was recorded as FIG. 8B.

FIGS. 3B(i)-3B(ii). A prewarmed suspension of macrophages in the complete medium (3.5×10⁵ cells/mL, 1 mL, 37° C.) supplemented with LPS (1 μg/mL) was added onto an array of PNIPAM microparticles on a bare glass coverslip in a device. The macrophages were incubated (5% CO₂, 37° C.). After 3 h, Hoechst 33342 was added into the culture to reach a concentration of 1 μg/mL and incubated (5% CO₂, 37° C.) for 30 min. LysoView 488 was added to culture to reach 1× LysoView 488 concentration and incubated (5% CO₂, 37° C.) for 30 min. The culture was washed with 22° C. PBS and then imaged to obtain FIG. 3B(i). To obtain FIG. 3B(ii), the 37° C. complete medium in a culture was replaced with 0° C. PBS immediately (within 1 min) after the culture was taken out of the incubator. The culture was then placed on ice and maintained for 2 min before being imaged.

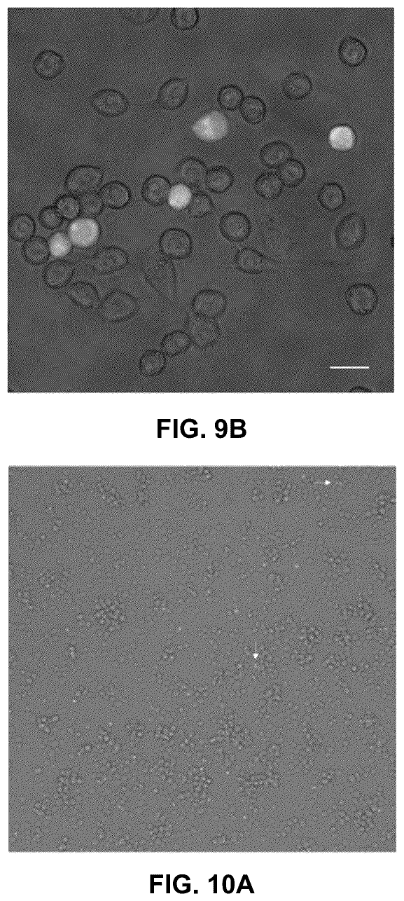

FIGS. 3C(i)-3C(ii) and 9A-9B. PNIPAM-fluorescein microparticles were printed on two PVA-coated glass coverslips. A chamber was created on one of the coverslips and sterilized with UV as described above. The microparticle-carrying PVA film on the other coverslip was detached from the coverslip, sterilized with UV as described above, and placed in the chamber. A prewarmed suspension of macrophages in the complete medium (3.5×10⁵ cells/mL, 1 mL, 37° C.) supplemented with LPS (1 μg/mL) was added into the chamber. The macrophages were incubated (5% CO₂, 37° C.) for 24 h. The culture was washed with 37° C. PBS three times and imaged in 37° C. PBS to obtain FIG. 3C(i). The 37° C. PBS in the culture was then replaced with 0° C. PBS when the culture was kept on the microscope stage. After 2 min, the culture was imaged to obtain FIG. 3C(ii). The images of FIGS. 9A-9B were taken from a different sample following the same procedure as mentioned above.

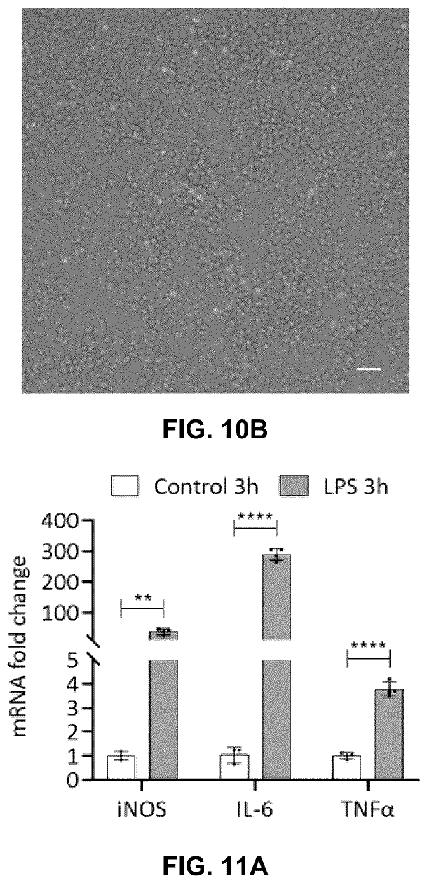

FIGS. 10A-10B. A prewarmed suspension of macrophages in the complete medium (3.5×10⁵ cells/mL, 1 mL, 37° C.) supplemented with LPS (1 μg/mL) was added onto an array of PNIPAM microparticles on a bare glass coverslip in a device and incubated 5% CO₂ and 37° C. After 3 h, the culture was washed with 22° C. PBS three times, kept in 22° C. PBS, and imaged to obtain FIG. 10A. The macrophages were further incubated (5% CO₂, 37° C.) for 24 h. To obtain FIG. 10B, the complete medium was replaced with 0° C. PBS immediately (within 1 min) after the culture was taken out of the incubator. The culture was then placed on ice and maintained for 2 min before being imaged.

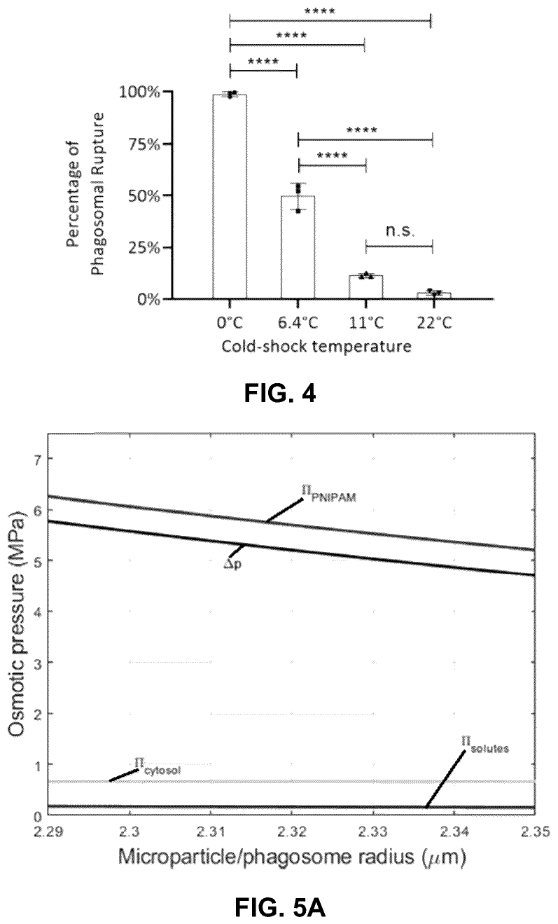

FIG. 4. A prewarmed suspension of macrophages in the complete medium (3.5×10⁵ cells/mL, 1 mL, 37° C.) supplemented with LPS (1 μg/mL) was added onto an array of PNIPAM microparticles on a bare glass coverslip in a device. The macrophages were incubated (5% CO₂, 37° C.). After 3 h, the complete medium was replaced with 0, 6.4, 11, and 22° C. PBS, respectively. After 2 min, the culture was imaged using a 10×objective lens. At least six images of representative fields were taken for each sample.

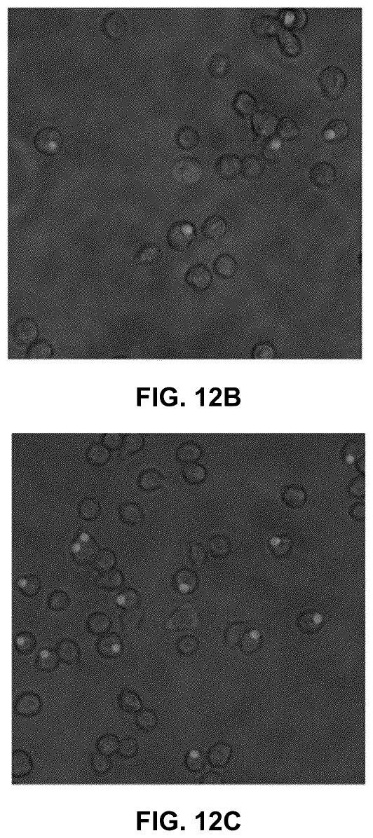

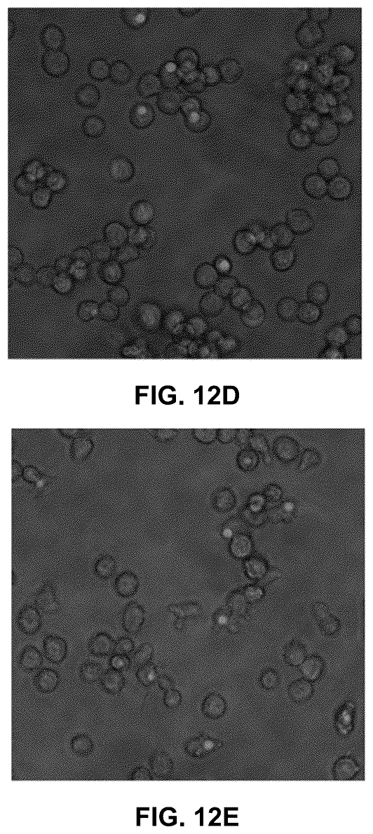

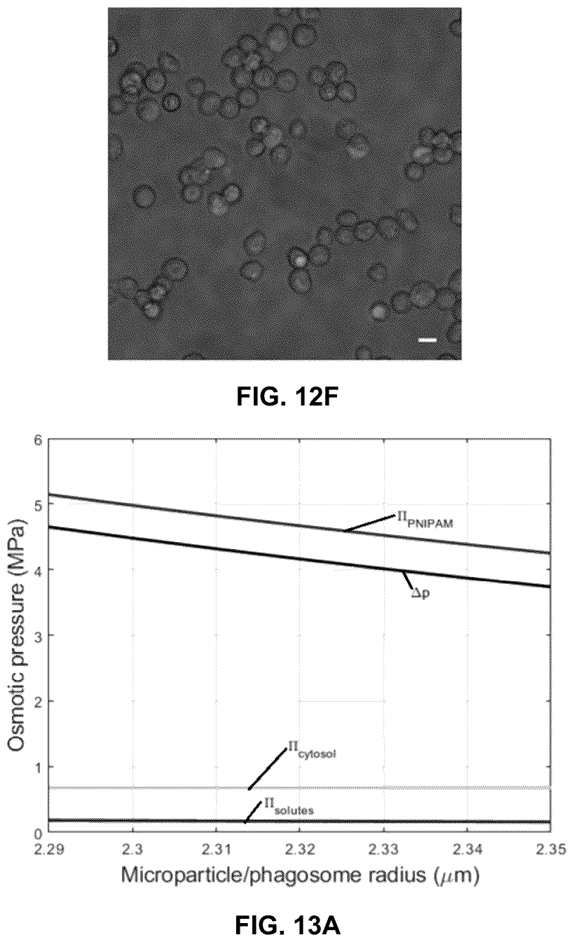

### Effects of Factors on Phagosomal Rupture at 22° C. (FIGS. 6 and 12A-12F)

Incubation of macrophages with microparticles. A square array (1 cm×1 cm) of PNIPAM microparticles was printed on a glass coverslip as mentioned above. A chamber was created on the coverslip to enclose PNIPAM microparticles, and the assembled device was sterilized with UV as described above. A prewarmed suspension of macrophages in the complete medium (3.5×10⁵ cells/mL, 1 mL, 37° C.) supplemented with LPS (1 μg/mL) was added into a chamber and incubated (5% CO₂, 37° C.) for the specified period.

Control. Macrophages and PNIPAM microparticles were incubated as described above. After 3 h, the complete medium was replaced with PBS (1 mL, 22° C.) and maintained for 2 min before being imaged.

Hypotonic shock. 0.6×PBS was prepared by mixing PBS with H₂O (volume ratio=6:4). The macrophages and PNIPAM microparticles were incubated as described above. After 3 h, the complete medium was replaced with the 0.6×PBS (1 mL, 22° C.) and maintained for 2 min before being imaged.

Chloroquine. The macrophages were incubated with PNIPAM microparticles as described above. After 2.5 h, chloroquine (10 mM in DMEM) was added to the culture to reach 100 μM and further incubated (5% CO₂, 37° C.) for 30 min. The complete medium was replaced with PBS (1 mL, 22° C.) and maintained for 2 min before being imaged.

Tetrandrine. The macrophages were incubated with PNIPAM microparticles as described above. After 2.5 h, tetrandrine (5 mM in DMSO) was added to the culture to reach 5 μM and further incubated (5% CO₂, 37° C.) for 30 min. The complete medium was replaced with PBS (1 mL, 22° C.) and maintained for 2 min before being imaged.

Colchicine. The macrophages were incubated with PNIPAM microparticles as described above. After 3 h, colchicine (2.5 mM in DMEM) was added to the culture at 5 μM and further incubated (5% CO₂, 37° C.) for 3 h. The complete medium was replaced with PBS (1 mL, 22° C.) and maintained for 2 min before being imaged.

LLOMe. The macrophages were incubated with PNIPAM microparticles as described above. After 2.5 h, LLOMe (10 mM in DMEM) was added to the culture at 1 mM and further incubated (5% CO₂, 37° C.) for 30 min. The complete medium was replaced with PBS (1 mL, 22° C.) and maintained for 2 min before being imaged.

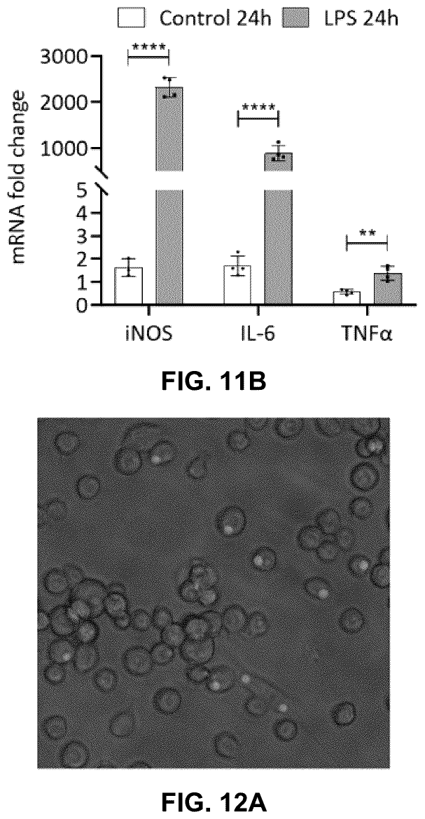

Real-Time qRT-PCR (FIGS. 11A-11B)

The total RNA was extracted from the macrophages using the TRIzol reagent (Invitrogen, 15596026) according to the product's user guide. The quantity and quality of the RNA were measured using a ND-1000 spectrophotometer (NanoDrop Technologies, Wilmington, DE, USA). Reverse transcription was then done following the manual of the qScript cDNA SuperMix (Quantabio, 95048-500) with 400 ng of RNA as the template in a 20 μL reaction system incubated in a Mastercycler nexus gradient (Eppendorf, Enfield, CT, USA). 1 μL/well cDNA was subjected to real-time qRT-PCR in a 20 μL reaction system using a PerfeCTa SYBR Green SuperMix (Quantabio, 101414-152) in a 96-well white plate (Bio-Rad, MLL9651) on a Bio-Rad CFX Connect Real-Time PCR System (Bio-Rad, Hercules, California, USA). The reactions were incubated at 95° C. for 5 min, followed by 40 cycles of 95° C. for 20 s, 60° C. for 10 s, and 72° C. for 30 s. All reactions were run in triplicates. The default threshold was used, and the Ct values were collected and averaged within triplicates. Then, the mean Ct values were subjected to the 2-AACt method to determine the relative expression level of mRNAs normalized to R-actin. Suitable primers were purchased from IDT (Coralville, Iowa, USA).

### Optical Microscopy

All optical micrographs were taken with an inverted Nikon Ti epifluorescence microscope equipped with an Andor iXon+885 EMCCD camera. A Nikon B-2E/C filter set was used for PNIPAM-fluorescein and LysoView. A Nikon G-2E/C filter set was used for the fluorescent nanoparticles. A Nikon UV-2E/C filter set was used for Hoechst 33342.

### Statistical Analysis

All data that were statistically analyzed were obtained from three or four independent experimental repeats. For each repeat, multiple macrophages or microparticles were sampled to calculate its mean. For each experiment, the mean of the sample means and the standard error of the sample means (SEMs) were calculated. Data are expressed as mean±SEM. Student's t-test (two-sample unequal variance, two-tailed) was performed to compare the differences of the data using Microsoft Excel. One-way ANOVA test and Tukey's post hoc comparisons were conducted using GraphPad Prism. The differences were considered as statistically significant at p<0.05 (denoted as *), very significant at p<0.01 (denoted as **), highly significant at p<0.001 (denoted as ***), and extremely significant at p<0.0001 (denoted as ****). The difference was regarded as not significant (ns) at p>0.05.

### Example 2: Results and Discussion

### Characterization of Microparticles

Two types of microparticles were fabricated and used as phagocytic objects in this study. One type was made of a commercial uncrosslinked linear PNIPAM with a molecular weight of 40 kDa. To fluorescently label the microparticles, red fluorescent sulfate-modified polystyrene nanoparticles with a diameter of 100 nm were dispersed in the microparticles. A microcontact-printing method that was previously developed was modified to fabricate the microparticles. Specifically, a thin film of PNIPAM containing the nanoparticles was spin-coated onto a PDMS stamp carrying an array of 5 μm-radius pillars using an aqueous solution of PNIPAM and the nanoparticles. The resultant film on the pillars was then transferred onto a glass coverslip via a conformal contact, generating an array of circular microparticles over a centimeter-wide printing area. The microparticles had an average radius of 5.07±0.03 μm (from 60 microparticles in three samples). The microparticles were visible in both bright-field and fluorescence images (FIGS. 2A(i)-2A(iii)), indicating that the microparticles were composed of both PNIPAM and the nanoparticles. Moreover, the microparticles were highly uniform in size, shape, and fluorescence intensity. To further characterize the morphology, the microparticles were imaged with SEM (FIGS. 2B(i)-2B(ii)), revealing a disk-like shape with a thickness much smaller than the diameter. To determine the thickness of the microparticles, the same microparticles as mentioned above were printed on a thin film of PVA, and the resulting film was manually cut with a razor blade across the microparticle array (FIG. 2C(i)). Using the cross-sectional images of the cut-through microparticles (FIG. 2C(ii)), the average thickness of the microparticles was determined to be 1.2±0.1 μm (from 42 microparticles in three samples). To demonstrate that the microparticles were not soluble in an aqueous environment at 37° C., the microparticles were printed on a PVA-coated glass coverslip and then soaked in PBS at 37° C. for 7 d. The PVA film was used to release the microparticles from the coverslip surface because it is soluble in water. As shown in FIGS. 2D(i)-2D(iii), the soaked microparticles were circular in shape and highly uniform in size. The random distribution of the soaked microparticles indicates that they were indeed released from the coverslip surface. Moreover, some microparticles were moving when imaged, suggesting that the soaked micro-particles had a spherical shape. It was hypothesized that the original disk-shaped microparticles were released from the coverslip surface upon addition of 37° C. PBS and that the released microparticles became hydrated and transformed into a spherical shape driven by surface area minimization during the period of soaking. This hypothesis is consistent with the observation that the soaked microparticles, with an average radius of 2.3±0.3 μm (from 30 microparticles in 3 samples), were much smaller than the lateral radius of the original disk-shaped microparticles. Moreover, the soaked microparticles exhibited bright fluorescence, indicating that the nanoparticles were trapped in the hydrated PNIPAM matrix of the microparticles. To examine if the microparticles were soluble in an aqueous solution at a lower temperature, the micro-particles printed on a PVA-coated glass coverslip were released with a prewarmed complete cell culture medium (37° C.) and further incubated for 3 h at 37° C. The microparticles in the complete medium were then imaged in the ambient environment at 22° C. The temperature of the complete medium gradually decreased (FIG. 2E(i)). The microparticles initially exhibited a compact and bright appearance at 35.6° C. (FIG. 2E(ii)), swelled in size and dimmed in fluorescence at 29° C. (FIG. 2E(iii)), and then quickly (within 1 min) and almost simultaneously disappeared at 28.5° C. (FIG. 2E(iv)). By repeating this experiment three times, the temperature at which the microparticles just disappeared was determined to be 28.8±0.3° C. This type of microparticles were used throughout this study unless otherwise noted.

The other type of microparticles used in this study were made of a PNIPAM derivative, which was synthesized by randomly copolymerizing N-isopropylacrylamide and fluorescein-O-acrylate as described previously. The copolymer, named PNIPAM-fluorescein hereafter, had a N-isopropylacrylamide-to-fluorescein mole ratio of 98:1 and a molecular weight of 5.12 kDa and was intrinsically green fluorescent. Microparticles made of PNIPAM-fluorescein alone were fabricated as described previously.

### Phagosomal Rupture and Effect of Cold-Shock Temperature

Phagocytosis of the PNIPAM microparticles was induced by adding macrophages in the complete medium at 37° C. to the microparticles printed on a glass coverslip unless otherwise noted. After incubation at 37° C. for 3 h, both macrophages, identified based on their bright-field morphology and Hoechst 33342-stained nuclei, and microparticles, visible in both the bright-field mode and the fluorescence mode, were imaged (FIG. 7). While some microparticles (6 in FIG. 7, indicated by arrows) were colocalized with the macrophages, most (36 in FIG. 7) were not colocalized with the macrophages and maintained the arrayed pattern, indicating that incubation in the 37° C. medium did not release the microparticles from the coverslip surface. However, the microparticles shrunk in lateral radius to 3.1±0.2 μm (from 30 microparticles in 3 samples). The existence of the microparticles that were not colocalized with the macrophages indicates that the temperature of the culture was between 28.8 and 37° C. when the image was taken. As imaging proceeded approximately 13 min after the culture was taken out of the incubator, all microparticles that were not colocalized with the macrophages disappeared and all the remaining microparticles (indicated by arrows) were colocalized with macrophages as shown in FIG. 3A(i). It is believed that the non-colocalized microparticles dissolved as the temperature of the culture dropped below 28.8° C. The temperature of the culture shown in FIG. 3A(i) should thus be between room temperature (22° C.) and 28.8° C. Most importantly, existence of the remaining microparticles in FIG. 3A(i) indicates that they were enclosed in non-ruptured phagosomes. Otherwise, the microparticles should have disappeared due to the dissolution in the medium if they were outside of the macrophages, or they should have spread in the cytosol of the macrophages if they were inside the macrophages but not inside the phagosomes. One of the microparticles in FIG. 3A(i) is magnified as shown in FIG. 3A(ii), revealing that the microparticle was circular in shape and located between the nucleus and periphery of a macrophage.

To induce rupture of the phagosomes, the 37° C. complete medium was replaced with 0° C. PBS immediately after taking the culture out of the incubator and maintained the culture on ice. This treatment will be referred to as 0° C. cold shock. It is reported that exposure to 0° C. PBS does not raise the acidic pH in bacteria-containing phagosomes in live mouse primary macrophages and RAW264.7 macrophages, respectively, indicating that 0° C. alone does not rupture the phagosomes. FIG. 3A(iii) shows a typical culture after 0° C. cold shock, in which 13 macrophages (indicated by arrows) display strong fluorescence. One of the macrophages is magnified in FIG. 3A(iv). The distribution patterns of the fluorescence within the macrophages in FIG. 3A(iii) are starkly different from those in FIG. 3A(i). Specifically, the fluorescent areas in the arrow-indicated macrophages in FIG. 3A(iii) were much larger than those in FIG. 3A(i). Moreover, the fluorescent areas in FIG. 3A(iii) largely overlap with the cytoplasm of the macrophages. These two features are more clearly observed by comparing FIGS. 3A(ii) and 3A(iv) and strongly suggest that the PNIPAM microparticles in the phagosomes dissolved, ruptured the phagosomes, and spread into the cytoplasm as a result of the 0° C. cold shock. The fluorescent areas in FIG. 3A(iii) did not overlap with the nuclei, which is expected as the fluorescent nanoparticles had a diameter of 100 nm, and particles larger than 9 nm cannot enter nucleus via passive diffusion. Statistically, phagosomal rupture was found in 98.8±1.2% of 1260 macrophages that contained fluorescence of the nanoparticles in 3 samples. The process of phagosomal rupture took less than 1 min for most microparticle-containing phagosomes to rupture after 0° C. PBS was added to the culture. FIGS. 8A-8B shows the two images of the same area before and after rupture.

To further confirm that phagosomes were ruptured, the microparticle-containing phagosomes were labeled with LysoView 488 dye, which can accumulate in acidic phagosomes and emit green fluorescence. After dissolving microparticles that were not phagocytosed with 22° C. PBS, it was found that some phagocytosed microparticles, indicated by strong fluorescence, exhibited the strong green fluorescence of LysoView 488. As a representative image, FIG. 3B(i) shows 12 fluorescent circular objects with 9 of them also having a second color of fluorescence (indicated by arrows). The existence of fluorescence overlap indicates that the PNIPAM microparticles were enclosed in intact acidic phagosomes. By contrast, three fluorescent phagosomes in FIG. 3B(i) do not show strong second color fluorescence (marked by “a”), suggesting that the membrane of the three phagosomes was permeabilized in a way similar to lysosomal membrane permeabilization (LMP) induced by L-leucyl-L-leucine methyl ester (LLOMe). It is worth noting that one macrophage in FIG. 3B(i) underwent phagosomal rupture (marked by “R”). Statistically, among 1588 macrophages that contained fluorescence in four samples, only 1.4±0.4% of them had ruptured phagosomes. The 0° C. cold shock was also used as described above to induce phagosomal rupture in the macrophages with internalized PNIPAM microparticles and stained with LysoView 488. As shown in FIG. 3B(ii), the majority of microparticle-containing phagosomes ruptured as indicated by the appearance of diffuse fluorescence in the cytoplasm of the macrophages. Moreover, none of the macrophages that contained diffuse first color fluorescence had 5-6 μm-diameter circular phagosomes also showing a second fluorescence color, suggesting that the original phagosomes had ruptured. Note that there is a non-ruptured non-permeabilized phagosome (marked by “a”) in FIG. 3B(ii). Statistically, among 577 macrophages that contained fluorescence in three samples, 93.0±2.9% of them had ruptured phagosomes after being treated with the 0° C. cold shock. The results provide additional confirmation that PNIPAM microparticles were indeed internalized into the phagosomes and that a majority of the microparticle-containing phagosomes ruptured when treated with a 0° C. cold shock.

Red fluorescent nanoparticles were used to label the PNIPAM microparticles in the above experiments. To exclude the possibility that the nanoparticles caused phagosomal rupture, PNIPAM-fluorescein was used as the sole material to fabricate microparticles. The PNIPAM-fluorescein micro-particles were fabricated and incubated with the macrophages as described previously. After being cultured for 24 h, the culture was washed with and maintained in 37° C. PBS. It was observed that all the remaining PNIPAM-fluorescein micro-particles were colocalized with the macrophages (FIG. 3C(i)). It is noteworthy that the fluorescein moieties in the PNIPAM-fluorescein were conjugated to the PNIPAM chains through ester bonds. Zu et al. found that the fluorescein moieties can be released from the PNIPAM chains through cleavage of the ester bonds by esterase in endosomes of live cells, and the released fluorescein molecules enter the cytoplasm of the cells, exhibiting diffuse fluorescence. Fluorescein fluorescence was not observed in the cytosol of the three micro-particle-containing macrophages in FIG. 3C(i). Assuming that the phagosomes contained esterase, this result suggests that the released fluorescein molecules were confined in the phagosomes. FIG. 3C(ii) shows the same area as FIG. 3C(i) 2 min after replacing the medium with 0° C. PBS. The fluorescein fluorescence in the three microparticle-containing macro-phages in FIG. 3C(i) became diffuse in the macrophages, suggesting that the dissolved PNIPAM-fluorescein and possible free fluorescein molecules have spread in the cytoplasm of the macrophages due to phagosomal rupture. Two images similar to FIG. 3C(ii) from a different sample are shown in FIGS. 9A-9B. These results suggest that PNIPAM was responsible for phagosomal rupture caused by the PNIPAM microparticles labeled with the fluorescent nanoparticles.

By using a cold shock to induce phase transition of the microparticles in the phagosomes, this method allows a temporal control of phagosomal rupture. To demonstrate this capability, the macrophages were incubated with the microparticles in the complete medium at 37° C. for 3 h, removed any non-phagocytosed microparticles by washing the culture with 22° C. PBS, and further incubated the macro-phages in the complete medium at 37° C. for 24 h. Finally, the macrophages were treated with a 0° C. cold shock. The washing step ensured that all phagosome-enclosed micro-particles had been within phagosomes for at least 24 h before the cold shock. As expected, a low percentage of phagosomal rupture (3±1% of 895 macrophages in 3 samples) was observed right after washing the culture with the 22° C. PBS, and a high percentage of phagosomal rupture (99.9±0.2% of 617 macrophages in 3 samples) was observed after the 0° C. cold shock. A representative pair of images of the macrophages after the washing step and the cold shock respectively are shown in FIGS. 10A-10B. This result demonstrates that this method can induce phagosomal rupture at any time after complete phagocytosis of microparticles, making it useful to study whether a phagosome's ability to resist rupture is dependent on different stages of its development, which is a highly dynamic process.

LPS was added to the culture in all experiments using macrophages. While LPS is typically used to polarize macrophages toward a pro-inflammatory phenotype, it was used here to promote RAW264.7 macrophages to adopt a flattened morphology. Such a morphology is desirable for visualizing intracellular structures. However, to provide a more comprehensive characterization of the effects of LPS on the macrophages, real-time quantitative reverse transcription polymerase chain reaction (real-time qRT-PCR) was used to measure the mRNA levels of inducible nitric oxide synthase (iNOS), interleukin-6 (IL-6), and tumor necrosis factor α (TNFα) in the macrophages treated with LPS without microparticles. FIGS. 11A-11B show that all the three mRNAs were significantly upregulated at both 3 and 24 h, confirming that the macrophages were in a pro-inflammatory phenotype. This result is consistent with previous findings that levels of iNOS, IL-6, and TNFα mRNAs were significantly increased in RAW264.7 macrophages treated with LPS. Most importantly, this result indicates that the LPS-treated microparticle-containing macrophages were likely to have a pro-inflammatory phenotype.

The effect of temperature on phagosomal rupture was next studied. The percentage of phagosomal rupture, which is defined as a percentage of macrophages containing ruptured phagosomes among macrophages containing either ruptured or non-ruptured microparticle-containing phagosomes, was determined at four different cold-shock temperatures. FIG. 4 shows that the percentages of phagosomal rupture were 98.8 1.2, 49.8 6.4, 11.3±1.0, and 3.0±1.1% at 0, 6.4, 11, and 22° C., respectively. It is clear that the percentage of phagosomal rupture decreased with the increase of the cold-shock temperature. Statistical analysis with one-way ANOVA and Tukey's post hoc test reveals that pairwise differences are significantly different except between 11 and 22° C. (Table 1).

TABLE 1
Tukey's post hoc test of effect of cold-shock
temperature on percentage of phagosomal rupture

| Comparison pair | P value |
|---|---|
| 0° C. vs 11° C. | 4.18 × 10⁻⁷ |
| 0° C. vs 22° C. | 7.48 × 10⁻⁹ |
| 0° C. vs 6.4° C. | 4.25 × 10⁻⁹ |
| 6.4° C. vs 11° C. | 2.88 × 10⁻⁶ |
| 6.4° C. vs 22° C. | 6.14 × 10⁻⁷ |
| 11° C. vs 22° C. | 6.01 × 10⁻² |

### Theoretical Analysis

Based on the above results and existing knowledge about PNIPAM and phagocytosis, it is postulated that the following series of events occurred in the process of phagosomal rupture in this method. Initially, a PNIPAM microparticle became hydrated as it was soaked in the complete medium at 37° C. The microparticle was phagocytosed by a macrophage into its phagosome, and the microparticle was tightly enclosed by the phagosome. It is thus assumed that a microparticle-containing phagosome has the same size and shape as the microparticle before the cold shock. The microparticle changed from the water-insoluble hydrated state to a dissolved state when the culture was treated with a cold shock. This phase transition led to an increased osmotic pressure in the phagosome. Driven by the increased osmotic pressure, water flows from the cytosol into the phagosome, causing the microparticle and phagosome to swell. It is assumed that the phagosome and the microparticle maintained the same size and shape until the phagosome ruptured. Rupture occurred when the phagosomal membrane could not swell any bigger, and the critical rupture tension of the membrane was surpassed by the membrane tension induced by the osmotic pressure generated by the PNIPAM microparticle.

The osmotic pressure generated by PNIPAM, denoted as f-PNIPAM, can be calculated with the Flory-Huggins model as follows:

$${\prod }_{PNIPAM} = - \frac{{N}_{A} {k}_{B} T}{{v}_{s}} [ ϕ + ln ( 1 - ϕ ) + x {ϕ}^{2} ] \qquad Eq . ( 1 )$$

where N_{A} is Avogadro's number, k_{B} is the Boltzmann constant, T is the absolute temperature, vₛ is the molar volume of water, ϕ is the volume fraction of PNIPAM in a PNIPAM microparticle soaked in the complete medium, and χ is the Flory-Huggins polymer-solvent interaction parameter. χ in eq 1 is further expressed as

χ=½−A(1−θ/T)+Cϕ+Dϕ² Eq. (2)

where θ is the theta temperature and A, C, and D are constants determined by fitting the experimental data. It is worth noting that these microparticles resemble PNIPAM microgels, which are microscopic particles made of chemically crosslinked PNIPAM, in size and composition. PNIPAM microgels have been widely studied with the Flory-Rehner model, which combines the Flory-Huggins model with an elastic contribution to the osmotic pressure from the chemically crosslinked chains. The disclosed PNIPAM microparticles are different from the PNIPAM microgels in that the PNIPAM chains in these microparticles are not chemically crosslinked. The elastic contribution to the osmotic pressure in the Flory-Rehner theory is therefore not considered in this analysis.

In addition to water, small ions can partition between a macroscopic collapsed PNIPAM gel and an aqueous solution of the ions. It is assumed that other solutes such as glucose have the same property. The PNIPAM microparticles were soaked in the complete medium before being phagocytosed. It is assumed that small solutes including ions and non-charged small molecules in the complete medium partitioned between the PNIPAM microparticles and the medium. The solutes in the microparticles can thus generate osmotic pressure in the phagosomes. It is assumed that the osmotic pressure generated by the solutes in the phagosome, denoted as Πₛₒₗᵤₜₑₛ, can be approximated by the Morse equation

Πₛₒₗᵤₜₑₛ=M′ₛₒₗᵤₜₑₛRT Eq. (3)

where M′ₛₒₗᵤₜₑₛ is the molarity of the solutes in the phagosome with volume of PNIPAM excluded from the total volume of the phagosome, R is the ideal gas constant, and T is the absolute temperature.

It is assumed that the molarity of the solutes in the total volume of the phagosome before the cold shock, denoted as M_{solutes,0}, is related to the molarity of the complete medium, M_{medium}, through a partition coefficient K as

$$K = \frac{{M}_{solutes , 0}}{{M}_{medium}} \qquad Eq . ( 4 )$$

The total pressure difference, Δp, across the phagosomal membrane is the difference between the sum of osmotic pressures contributed by PNIPAM and solutes inside the phagosome and the osmotic pressure of the cytosol, Π_{cytosol}. It is expressed as

Δp=Π_{PNIPAM}+Πₛₒₗᵤₜₑₛ−Π_{cytosol} Eq. (5)

It is assumed that Π_{cytosol} can also be approximated by the Morse equation

Π_{cytosol}=M_{cytosol}RT Eq. (6)

where M_{cytosol} is the molarity of the cytosol. It is worth noting that a linear relationship between Π_{cytosol} and T has been reported.

Since DMEM supplemented with 10% FBS is an isotonic solution used for culturing mammalian cells, it is assumed

M_{medium}=M_{cytosol} Eq. (7)

The Young-Laplace equation is commonly used to relate the pressure difference across a membrane of a spherical membrane-bound vesicle such as endosomes and surface tension, y, in the membrane. The equation is as follows

Δp=2γ/r Eq. (8)

where r is the radius of the phagosome in this work.

Since the plasma membrane of a mammalian cell under a tensile stress can undergo area expansion and the phagosomal membrane is mainly derived from the plasma membrane, it is assumed that the ruptured phagosomes in this work underwent area expansion before rupture occurs. It is further assumed that the phagosomes maintained a spherical shape during the expansion and only water entered the expanding phagosomes. By denoting the radius of the microparticle as well as the phagosome at the beginning time of cold shock as r₀ and the corresponding volume fraction of PNIPAM as ϕ0, an expression of ϕ is obtained as

$$ϕ = {ϕ}_{0} \frac{{r}_{0}^{3}}{{r}^{3}} \qquad Eq . ( 9 )$$

M′ₛₒₗᵤₜₑₛ is expressed as

$${M}_{solutes}^{′} = {M}_{solutes , 0} \frac{{r}_{0}^{3}}{{r}^{3} - {ϕ}_{0} {r}_{0}^{3}} \qquad Eq . ( 10 )$$

Combining eqs 1-10 yields

$$γ = - \frac{{N}_{A}}{{v}_{s}} {k}_{B} T \{ {ϕ}_{0} \frac{{r}_{0}^{3}}{{r}^{3}} + ln ( \text{} 1 - {ϕ}_{0} \frac{{r}_{0}^{3}}{{r}^{3}} ) + [ \text{} \frac{1}{2} - A ( 1 - \frac{θ}{T} ) + C ( {ϕ}_{0} \frac{{r}_{0}^{3}}{{r}^{3}} ) + {D ( {ϕ}_{0} \frac{{r}_{0}^{3}}{{r}^{3}} )}^{2} ] {( {ϕ}_{0} \frac{{r}_{0}^{3}}{{r}^{3}} )}^{2} \} \text{} \frac{r}{2} + KM \frac{{r}_{0}^{3}}{{r}^{3} - {ϕ}_{0} {r}_{0}^{3}} RT \frac{r}{2} - MRT \frac{r}{2} \qquad Eq . ( 11 )$$

Equation 11 provides a mathematical expression for surface tension in the phagosomal membrane as a function of radius of the phagosome and the cold-shock temperature.

Since the plasma membrane of a mammalian cell can undergo up to 5% area expansion and a lipid bilayer being stretched can withstand a tension up to 10 mN/m before being ruptured, it is assumed that the phagosomes in this work can also withstand 5% area expansion and 10 mN/m tension. It is further assumed that the microparticles in the phagosomes had a spherical shape with a radius of 2.29 μm before the cold shock, that is, r₀=2.29 μm. A 5% area expansion of the phagosome corresponds to a radius of 2.35 μm, which is denoted as re, that is, rₑ=2.35 μm. Lopez and Richtering determined the volume fraction of PNIPAM in collapsed microgels in water as 0.44.9 It is assumed that these microparticles had this volume fraction before they were internalized into phagosomes, that is, ϕ₀=0.44. Lopez and Richtering also determined θ, A, C, and D in eq 2: θ=30.6° C., A=−2, C=0.32, and D=0.24. Note that the effects of the fluorescent nanoparticles in the microparticles are ignored. It is believed that this is acceptable because the weight fraction of the nanoparticles in the dry microparticles is very low (˜0.67%). The molarity of DMEM has been reported as 0.29 M. It is assumed that the complete medium, which was DMEM supplemented with 10% FBS and antibiotics and L-glutamine, had the same molarity, that is, M_{medium}=0.29 M. Kawasaki et al. found that the partition coefficient, K, for Na⁺ and Cl⁻ ions and collapsed PNIPAM gel is ˜0.15 in 0.3 M NaCl at 40° C. K=0.15 is thus adopted in this analysis.

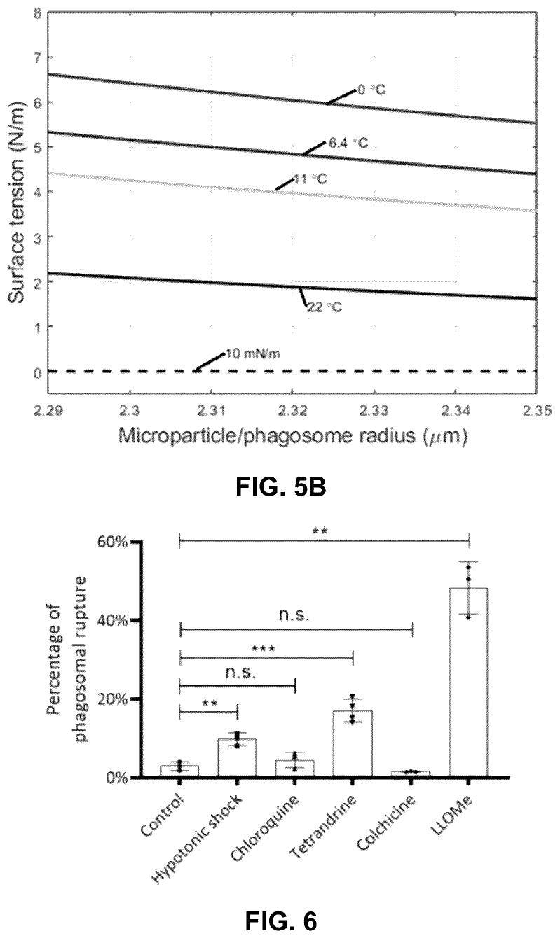

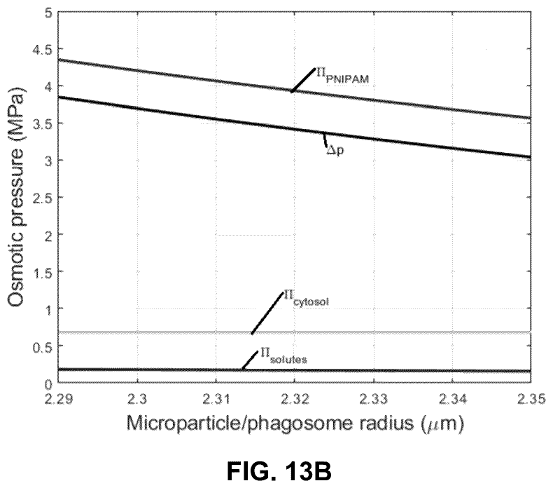

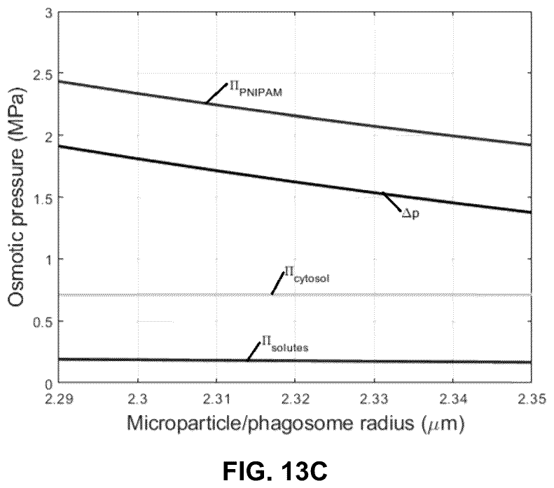

By using the above equations and parameters, Π_{PNIPAM} versus r is plotted, Πₛₒₗᵤₜₑₛ versus r, Π_{cytosol}, versus r, and Δp versus r from r₀=2.29 μm to rₑ=2.35 μm at 0° C. in FIG. 5A. The profiles reveal that Π_{PNIPAM} decreases with the increase of r, but it is an order of magnitude higher than Πₛₒₗᵤₜₑₛ or Π_{cytosol} from r₀ to re, indicating that Π_{PNIPAM} plays a dominant role in determining the osmotic pressure difference across the phagosomal membrane. The Δp vs r profile shows that a 4.7 MPa osmotic pressure difference across the phagosomal membrane exists at rₑ=2.35 μm. The plots at 6.4, 11, and 22° C. are shown in FIGS. 13A-13C, exhibiting the same pattern as in FIG. 5A. γ versus r from r₀=2.29 μm has also been plotted to rₑ=2.35 μm at 0, 6.4, 11, and 22° C. using eq 11 in FIG. 5B. It shows that y decreases as the phagosome swells at all temperatures. At any radius including r=2.35 μm, the surface tension decreases with the increase of the cold-shock temperature. This pattern is qualitatively consistent with the experimental result that the percentage of phagosomal rupture decreased with the increase of the cold-shock temperature (FIG. 4). However, the surface tension at all temperatures and rₑ=2.35 μm are 2 orders of magnitude higher than the critical rupture tension of a lipid bilayer, suggesting that all microparticle-containing phagosomes should have been ruptured at the temperatures. While this roughly agrees with the 98.8% rupture percentage at 0° C., it is inconsistent with the much lower rupture percentages at 6.4, 11, and 22° C. This inconsistency suggests the existence of unknown mechanisms used by the phagosomes to resist rupture.

### Effects of Various Factors on Phagosomal Rupture

It was next sought to further investigate the mechanism and demonstrate the applications of this method. Building on these findings that a 22° C. cold shock induced a low percentage of phagosomal rupture (as depicted in FIG. 4), it was assumed that this percentage could be increased when the osmotic pressure difference across the phagosomal membrane was increased or the critical rupture tension of the phagosomal membrane was decreased. Six factors were identified that could potentially increase the osmotic pressure difference across the phagosomal membrane or decrease the critical rupture tension of the phagosomal membrane. For each factor, an experiment was conducted to study its effect. In each experiment, PNIPAM microparticles were initially incubated with macrophages at 37° C. for 3 h, and phagosomal rupture was finally induced by a 22° C. cold shock. Exposure of the macrophages to each factor was conducted either during or before the cold shock. The percentage of phagosomal rupture was determined for each experiment and compared with that obtained from a control experiment, in which PNIPAM microparticles were incubated with macrophages at 37° C. for 3 h, followed by a 22° C. cold shock. A representative image of the macrophages in the control experiment following the 22° C. cold shock is shown in FIG. 13A.

The first factor studied was hypotonic shock, which is commonly generated with a 0.6×cell culture medium and traditionally combined with a prior exposure of cells to a hypertonic solution to induce endosomal rupture. Here, the microparticle-containing macrophages were simply exposed to 22° C. 0.6×PBS. Assuming that treatment would simultaneously decrease the osmotic pressure in the cytosol and cause the microparticles in the phagosomes to become soluble, it was speculated that the osmotic pressure difference across the membrane of a microparticle-containing phagosome would increase. As a result, an increased percentage of phagosomal rupture should be obtained if the PNIPAM microparticles ruptured the phagosomes by increasing the intra-phagosomal osmotic pressure. As shown in FIG. 6 and Table 2, the percentage of phagosomal rupture caused by the 22° C. 0.6×PBS treatment is significantly higher than that caused by the 22° C. 1×PBS treatment. A representative image of the macrophages following the 22° C. 0.6×PBS treatment is shown in FIG. 13B. This result supports the notion that an increased intra-phagosomal osmotic pressure is responsible for rupturing the microparticle-containing phagosomes in the experiments using the cold isotonic PBS.

TABLE 2
Student's t-test of effect of various factors on percentage
of phagosomal rupture at 22° C.^{a, b}

| Factor | P-value |
|---|---|
| Hypotonic shock | 0.00577 |
| Chloroquine | 0.32134 |
| Tetrandrine | 0.00089 |
| Colchicine | 0.15687 |
| LLOMe | 0.00598 |

^{a}Two sample unequal variance, 2-tailed
^{b}All factors are compared to the control

The second factor studied was chloroquine, which is a small-molecule drug traditionally used for treating malaria. Chloroquine is lysosomotropic, meaning that it can accumulate in acidic subcellular compartments such as lysosomes. It was thus assumed that chloroquine could accumulate in the microparticle-containing phagosomes. It is also reasonable to speculate that the accumulation could result in an increase in osmotic pressure inside the phagosomes. IT was tested if chloroquine could affect the percentage of phagosomal rupture. The result (FIG. 6 and Table 2) reveals that the condition used in this study (100 μM chloroquine and 30 min incubation) did not induce a significant effect, suggesting that chloroquine did not increase the osmotic pressure inside the phagosomes. A representative image of the macrophages following the 22° C. cold shock is shown in FIG. 13C.

The third factor studied was tetrandrine, which is a potent inhibitor of two-pore channels (TPCs). Freeman et al. found that the Na+ ion inside macropinosomes in macrophages exited the macropinosomes via the TPCs, osmotically driving the macropinosomes to shrink, and this process could be inhibited by tetrandrine. Since TPCs are expressed at high levels in macrophages including the RAW264.7 cells used in this study, it was assumed that TPCs could reduce the osmotic pressure inside the microparticle-containing phagosomes. It was thus hypothesized that treating the microparticle-containing phagosomes with tetrandrine would lead to an increased osmotic pressure difference across the phagosomal membrane by decreasing the efflux of Na+ from the phagosomes compared to the control and consequently an increase in the percentage of phagosomal rupture. FIG. 6 and Table 2 show that the treatment indeed significantly increased the percentage of phagosomal rupture. This result proves the hypothesis and also supports the notion that Na+ was present in the microparticle-containing phagosomes as was postulated in the Theoretical Analysis section.

The fourth factor studied was colchicine, which is a microtubule-depolymerizing agent. Certain fungi can grow in a macrophage phagosome, and the phagosome can enclose the fungus without being ruptured through its fusion with lysosomes. Using RAW264.7 macrophages, Westman et al. found that the phagosome-lysosome fusion could be inhibited by colchicine, and the treatment led to phagosomal rupture. It was hypothesized that treating the microparticle-containing phagosomes with colchicine for 3 h would lead to an increased osmotic pressure difference across the phagosomal membrane by inhibiting the phagosome-lysosome fusion compared to the control and consequently increase the percentage of phagosomal rupture. The result (FIG. 6 and Table 2) shows that the effect of colchicine was insignificant, suggesting that the phagosome-lysosome fusion did not decrease the osmotic pressure in the microparticle-containing phagosomes under the experimental condition tested here.

The last factor studied was L-leucyl-L-leucine O-methyl ester (LLOMe). LLOMe can accumulate in lysosomes and be polymerized by the lysosomal enzyme cathepsin C, and the formed polymer disrupts the lysosomal membrane. Repnik et al. found that LLOMe could cause rapid LMP in HeLa cells without rupturing the lysosomes. It was hypothesized that treating the microparticle-containing phagosomes with LLOMe would reduce the critical rupture tension of the phagosomal membrane by disrupting its structural integrity and consequently increase the percentage of phagosomal rupture. FIG. 6 and Table 2 show that the treatment indeed significantly increased the percentage of phagosomal rupture. This result proves the hypothesis and suggests that LLOMe reduces the critical rupture tension of the phagosomal membrane.

### DISCUSSION

The disclosed phagosome-rupturing method features the use of osmotic pressure as the sole mechanism to rupture phagosomes. Since osmotic pressure has been used to rupture endosomes in the two existing methods, it is worthwhile to compare them with this method. One of the existing methods uses nanoparticles made of polymers that can swell upon a temperature drop. The nanoparticles are first internalized into endosomes in cells through endocytosis, and then the cells are cooled to induce swelling of the nanoparticles to consequently burst the endosomes. However, this method has never been used specifically to rupture phagosomes. Moreover, the polymers used for constructing the nanoparticles, which were crosslinked Pluronic F127 and poly(ethylene imine) (PEI), oligo(lactic acid)-b-Pluronic F127-b-oligo(lactic acid), and poly(L-lysine)-g-poly(ethylene glycol), have not been extensively studied from a perspective of theoretical modeling. The other method uses nanoparticles made of cationic polymers represented by PEI. It is originally believed that these polymers, once internalized into endosomes, can osmotically rupture them through the so-called proton sponge effect. However, recent studies indicate that the local interactions between the polymers and the endosomal membrane probably play an essential role in disrupting the membrane. This complicated mechanism renders theoretical modeling of this method difficult. Compared to these existing methods, only this method possesses a demonstrated ability to rupture phagosomes and can be readily modeled with a well-established polymer-physics theory.

The disclosed phagosome-rupturing method is a valuable tool for studying the mechanisms of phagosomal rupture in macrophages. Out findings suggest the existence of a mechanism that prevents the microparticle-containing phagosomes from rupturing over the entire range of cold-shock temperatures, including 0° C. By assuming that energy-dependent cellular activities are significantly inhibited at 0° C., it is speculated that the rupture-resisting mechanism at 0° C. is associated with the lipid composition of the phagosomal membrane or proteins attached to the cytosolic side of the phagosomal membrane.

Observing that the percentage of phagosomal rupture decreased as the cold-shock temperature increased, it is speculated that energy-dependent cellular activities, presumed to be more active at higher temperatures, can effectively resist phagosomal rupture. Furthermore, these results have shown that chloroquine does not cause a significant increase in the osmotic pressure inside phagosomes, that the export of Na+ from the phagosomes reduces the osmotic pressure inside them, that the phagosome-lysosome fusion does not lead to a significant decrease in the osmotic pressure inside phagosomes, and that LLOMe reduces the critical rupture tension of the phagosomal membrane.

The disclosed phagosome-rupturing method can be extended in three directions. First, in addition to RAW264.7 macrophages, this method can be used to study phagosomal rupture in other macrophages such as microglia and tumor-associated macro-phages, which play critical roles in diseases such as Alzheimer's disease and cancer, respectively. This method may also be used to study the immunity of dendritic cells because phagosomal rupture is a major pathway for antigen cross-presentation. Second, the theoretical model can be further tested experimentally and refined. Experimental test of the model can be conducted using PNIPAM with a series of molecular weight and microparticles with a series of sizes. Refinement of the model can be performed by determining the parameters in Flory-Huggins theory for the PNIPAM used in this study at a condition that mimics the phagosomal environment and cold shock. The refined model may be used to quantitatively determine the fundamental biophysical properties of the phagosome membrane such as critical rupture tension in live macrophages. Furthermore, this method may stimulate interests in using other polymer-physics theories to explain the experimental results. Third, given the fact that the cytosolic delivery of nanoparticles into macrophages has been demonstrated in this study, this method can be used to deliver nanoparticles loaded with drugs or antigens into the cytosol of macrophages or dendritic cells for treating or preventing a wide range of diseases.

### CONCLUSIONS

A robust engineering method for inducing phagosomal rupture in live macrophages through a well-defined mechanism has been established. The method uses microfabricated micro-particles composed of uncrosslinked linear PNIPAM as the phagocytic objects and cold shock-induced phase transition of the microparticles to osmotically rupture the phagosomes. Theoretical analysis based on Flory-Huggins theory suggests that the increased osmotic pressure caused by the dissolved microparticles is responsible for phagosomal rupture, and there exists a cellular mechanism resisting phagosomal rupture. Using this method, the effects of chloroquine, tetrandrine, colchicine, and LLOMe on phagosomal rupture have been determined. This method holds the potential to be further developed into a valuable research tool or an effective clinical therapy.

### Example 3: Method for Treating Cancer and/or Other Diseases

Microparticles containing iron oxide nanoparticles are delivered into the cytosol of the macrophages extracted from a cancer patient. It is known that iron oxide nanoparticles can polarize macrophages toward a cancer-fighting phenotype by catalyzing certain chemical reactions in the cytosol of the cells. The macrophages are then infused back into the patient and expected to accumulate in the metastatic solid tumors in the patient. The iron oxide nanoparticles in the macrophages are expected to keep the macrophages in the cancer-fighting phenotype even the tumor microenvironment tends to convert the macrophages to a cancer-promoting phenotype. As a result, the macrophages can inhibit the growth of the tumors.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Anderson, N. R. et al, Macrophage-Based Approaches for Cancer Immunotherapy, Cancer Res. 81 (2021) 1201-1208.
2. Bian, H.; et al. MAPK/P38 Regulation of Cytoskeleton Rearrangement Accelerates Induction of Macrophage Activation by TLR4, but Not TLR3. Int. J. Mol. Med. 2017, 40, 1495-1503.
3. Bus, T.; et al. The Great Escape: How Cationic Polyplexes Overcome the Endosomal Barrier. J. Mater. Chem. B 2018, 6, 6904-6918.
4. Canton, J.; et al. The Receptor DNGR-1 Signals for Phagosomal Rupture to Promote Cross-Presentation of Dead-Cell-Associated Antigens. Nat. Immunol. 2021, 22, 140-153.
5. Chahine, N. O.; et al. Direct Measurement of Osmotic Pressure of Glycosaminoglycan Solutions by Membrane Osmometry at Room Temperature. Biophys. J. 2005, 89, 1543-1550.
6. Cheng, W.; et al. Specific Labelling of Phagosome-Derived Vesicles in Macrophages with a Membrane Dye Delivered with Microfabricated Microparticles. Acta Biomater. 2022, 141, 344-353.
7. Childs, E.; et al. Maintenance and Loss of Endocytic Organelle Integrity: Mechanisms and Implications for Antigen Cross-Presentation. Open Biol. 2021, 11, 210194.
8. Chollet, B. et al, Multiscale Surface-Attached Hydrogel Thin Films with Tailored Architecture, ACS Appl. Mater. Interfaces. 8 (2016) 11729-11738.
9. de Duve, C.; et al. Lysosomotropic agents. Biochem. Pharmacol. 1974, 23, 2495-2531.
10. Flannagan, R. S.; et al. The Cell Biology of Phagocytosis. Annu. Rev. Pathol. 2012, 7, 61-98.
11. Freeman, S. A.; et al. Lipid-Gated Monovalent Ion Fluxes Regulate Endocytic Traffic and Support Immune Surveillance. Science 2020, 367, 301-305.
12. Freeman, S. A.; et al. Resolution of Macropinosomes, Phagosomes and Autolysosomes: Osmotically Driven Shrinkage Enables Tubulation and Vesiculation. Traffic 2018, 19, 965-974.
13. Goldman, S. D.; et al. Mechanisms of Amine Accumulation in, and Egress from, Lysosomes. Bioanalysis 2009, 1, 1445-1459.
14. Görlich, D.; et al. Transport between the Cell Nucleus and the Cytoplasm. Annu. Rev. Cell Dev. Biol. 1999, 15, 607-660.
15. Guan, J.; et al. Fabrication of Polymeric Microparticles for Drug Delivery by Soft Lithography. Biomaterials 2006, 27, 4034-4041.
16. Guo, M.; et al. Cell Volume Change through Water Efflux Impacts Cell Stiffness and Stem Cell Fate. Proc. Natl. Acad. Sci. U.S.A. 2017, 114, E8618-E8627.
17. Guo, Z. et al, Biocompatibility and cellular uptake mechanisms of poly(N-isopropylacrylamide) in different cells, Journal of Bioactive and Compatible Polymers. 32 (2017).
18. Halperin, A.; et al. Poly(N-Isopropylacrylamide) Phase Diagrams: Fifty Years of Research. Angew. Chem., Int. Ed. 2015, 54, 15342-15367.
19. Hansen, D. V.; et al. Microglia in Alzheimer's Disease. J. Cell Biol. 2018, 217, 459-472.
20. Hornung, V.; et al. Silica Crystals and Aluminum Salts Activate the NALP3 Inflammasome through Phagosomal Destabilization. Nat. Immunol. 2008, 9, 847-856.
21. Huang, B.-P.; et al. Anti-Inflammatory Effects of Perilla Frutescens Leaf Extract on Lipopolysaccharide-Stimulated RAW264.7 Cells. Mol. Med. Rep. 2014, 10, 1077-1083.
22. Huttu, M.; et al. Effects of Medium and Temperature on Cellular Responses in the Superficial Zone of Hypo-Osmotically Challenged Articular Cartilage. J. Funct. Biomater. 2012, 3, 544-555.
23. Ip, W. K. E.; et al. Phagocytosis and Phagosome Acidification Are Required for Pathogen Processing and MyD88-Dependent Responses to Staphylococcus Aureus. J. Immunol. 2010, 184, 7071-7081.
24. Jaumouille, V.; et al. Physical Constraints and Forces Involved in Phagocytosis. Front. Immunol. 2020, 11, 1097.
25. Kawasaki, H.; et al. Partition of Salts between N-Isopropylacrylamide Gels and Aqueous Solutions. Langmuir 2000, 16, 1444-1446.
26. Klichinsky, M. et al, Human chimeric antigen receptor macrophages for cancer immunotherapy, Nat Biotechnol. 38 (2020) 947-953.
27. Lam, R. S.; et al. Unprimed, M1 and M2 Macrophages Differentially Interact with Porphyromonas Gingivalis. PLoS One 2016, 11, No. e0158629.
28. Lanzalaco, S. et al, Poly(N-isopropylacrylamide) and Copolymers: A Review on Recent Progresses in Biomedical Applications, Gels. 3 (2017).
29. Le Roux, A.-L.; et al The Plasma Membrane as a Mechanochemical Transducer. Philos. Trans. R. Soc., B 2019, 374, 20180221.
30. Lecoultre, M.; et al. Phagocytic Function of Tumor-Associated Macrophages as a Key Determinant of Tumor Progression Control: A Review. J. Immunother. Cancer 2020, 8, No. e001408.
31. Lee, S. et al, Macrophage-based cell therapies: The long and winding road, J Control Release. 240 (2016) 527-540.
32. Lee, S. H.; et al. Thermally Sensitive Cationic Polymer Nanocapsules for Specific Cytosolic Delivery and Efficient Gene Silencing of SiRNA: Swelling Induced Physical Disruption of Endosome by Cold Shock. J. Controlled Release 2008, 125, 25-32.
33. Lee, Y.; et al. Thermally Triggered Intracellular Explosion of Volume Transition Nanogels for Necrotic Cell Death. J. Controlled Release 2009, 135, 89-95.
34. Livak, K. J.; et al. Analysis of Relative Gene Expression Data Using Real-Time Quantitative PCR and the 2-AACT Method. Methods 2001, 25, 402-408.
35. Lopez, C. G.; et al. Does Flory-Rehner Theory Quantitatively Describe the Swelling of Thermoresponsive Microgels? Soft Matter 2017, 13, 8271-8280.
36. Lopez-Leon, T.; et al. Macroscopically Probing the Entropic Influence of Ions: Deswelling Neutral Microgels with Salt. Phys. Rev. E: Stat., Nonlinear, Soft Matter Phys. 2007, 75, 011801.
37. Louf, J.-F.; et al. Under Pressure: Hydrogel Swelling in a Granular Medium. Sci. Adv. 2021, 7, No. eabd2711.
38. Martinez, F. O.; et al. The M1 and M2 Paradigm of Macrophage Activation: Time for Reassessment. F1000Prime Rep. 2014, 6, 13.
39. Nagahama, K.; et al. Measurement of Osmotic Pressure in Aqueous Solutions of Poly(Ethylene Glycol) and Poly(N-Isopropylacrylamide). Fluid Phase Equilib. 1994, 96, 203-214.
40. Orecchioni, M.; et al. Macrophage Polarization: Different Gene Signatures in M1(LPS+) vs. Classically and M2(LPS-) vs. Alternatively Activated Macrophages. Front. Immunol. 2019, 10, 1084.
41. Park, E.-J.; et al. Capric Acid Inhibits NO Production and STAT3 Activation during LPS-Induced Osteoclastogenesis. PLoS One 2011, 6, No. e27739.
42. Pelton, R. et al, Poly(N-isopropylacrylamide) (PNIPAM) is never hydrophobic, J Colloid Interface Sci. 348 (2010) 673-674.
43. Pizarro-Cerdd, J.; et al. Manipulation of Host Membranes by the Bacterial Pathogens Listeria, Francisella, Shigella and Yersinia. Semin. Cell Dev. Biol. 2016, 60, 155-167.
44. Repnik, U.; et al. L-leucyl-L-leucine Methyl Ester Does Not Release Cysteine Cathepsins to the Cytosol but Inactivates Them in Transiently Permeabilized Lysosomes. J. Cell Sci. 2017, 130, 3124-3140.
45. Schild, H. G., Poly(N-isopropylacrylamide): experiment, theory and application, Progress in Polymer Science. 17 (1992) 163-249.
46. Shields, C. W. et al, Cellular backpacks for macrophage immunotherapy, Sci Adv. 6 (2020) eaaz6579.
47. Simeone, R.; et al. Phagosomal Rupture by Mycobacterium Tuberculosis Results in Toxicity and Host Cell Death. PLoS Pathog. 2012, 8, No. el 002507.
48. Swanson, J. A. Shaping Cups into Phagosomes and Macropinosomes. Nat. Rev. Mol. Cell Biol. 2008, 9, 639-649.
49. Thiele, D. L.; et al. Mechanism of L-Leucyl-L-Leucine Methyl Ester-Mediated Killing of Cytotoxic Lymphocytes: Dependence on a Lysosomal Thiol Protease, Dipeptidyl Peptidase I, That Is Enriched in These Cells. Proc. Natl. Acad. Sci. U.S.A. 1990, 87, 83-87.
50. Tseng, D.; et al. Anti-CD47 Antibody-Mediated Phagocytosis of Cancer by Macrophages Primes an Effective Antitumor T-Cell Response. Proc. Natl. Acad. Sci. U.S.A. 2013, 110, 11103-11108.
51. Uribe-Querol, E.; et al. Phagocytosis: Our Current Understanding of a Universal Biological Process. Front. Immunol. 2020, 11, 1066.
52. Vermeulen, L. M. P.; et al. Endosomal Size and Membrane Leakiness Influence Proton Sponge-Based Rupture of Endosomal Vesicles. ACS Nano 2018, 12, 2332-2345.
53. Vermeulen, L. M. P.; et al. The Proton Sponge Hypothesis: Fable or Fact? Eur. J. Pharm. Biopharm. 2018, 129, 184-190.
54. Westman, J.; et al. Lysosome Fusion Maintains Phagosome Integrity during Fungal Infection. Cell Host Microbe 2020, 28, 798-812.e6.
55. Won, Y.-Y.; et al. Missing Pieces in Understanding the Intracellular Trafficking of Polycation/DNA Complexes. J. Controlled Release 2009, 139, 88-93.
56. Xu, Y.-W.; et al. Toxoplasma ROP161/III Ameliorated Inflammatory Bowel Diseases via Inducing M2 Phenotype of Macrophages. World J. Gastroenterol. 2019, 25, 6634-6652.
57. Zheng, N.; et al. Effect of Phospholipidosis on the Cellular Pharmacokinetics of Chloroquine. J. Pharmacol. Exp. Ther. 2011, 336, 661-671.
58. Zou, G. et al, Temperature-Sensitive Poly(N-isopropylacrylamide)/Konjac Glucomannan/Graphene Oxide Composite Membranes with Improved Mechanical Property, Swelling Capability, and Degradability, International Journal of Polymer Science. 2018 (2018) e7906747.
59. Zu, G.; et al. Nanogels with Selective Intracellular Reactivity for Intracellular Tracking and Delivery. Chemistry 2020, 26, 15084-15088.
60. Zuhorn, I. S. et al. Nonbilayer Phase of Lipoplex-Membrane Mixture Determines Endosomal Escape of Genetic Cargo and Transfection Efficiency. Mol. Ther. 2005, 11, 801-810.

What is claimed is:

1. A method for introducing loaded polymeric microparticles into one or more phagocytic cells, the method comprising:
(a) incubating loaded polymeric microparticles at a first temperature and in a first medium with the one or more phagocytic cells, wherein, during incubation, the loaded polymeric microparticles are taken up by the phagocytic cells into phagosomes, wherein the loaded polymeric microparticles comprise one or more nanoparticles;
(b) transferring the phagocytic cells containing loaded polymeric microparticles to a second medium at a second temperature at which the loaded polymeric microparticles swell;
(c) incubating the phagocytic cells at the second temperature, wherein during incubation, the phagosomes rupture in the phagocytic cells; and
(d) transferring the phagocytic cells to a third medium at a third temperature, to produce the loaded microparticles in the phagocytic cells.

2. The method of claim 1, further comprising loading polymeric microparticles with one or more nanoparticles to form the loaded polymeric microparticles prior to step (a).

3. The method of claim 1, wherein the loaded polymeric microparticles comprise poly(N-isopropylacrylamide) (PNIPAM), a copolymer thereof, a derivative thereof, or any combination thereof.

4. The method of claim 3, wherein the copolymer of PNIPAM comprises PNIPAM-fluorescein.

5. The method of claim 3, wherein the PNIPAM, copolymer thereof, or derivative thereof, has a molecular weight of from about 1000 Da to about 300,000 Da.

6. The method of claim 1, wherein the loaded polymeric microparticles have an average particle diameter of from about 0.1 μm to about 20 μm.

7. The method of claim 1, wherein the one or more nanoparticles comprise an anti-cancer agent, a fluorescent molecule, a metal or metal oxide, a live microorganism, an inactivated microorganism, a component of an inactivated microorganism, a polysaccharide or derivative thereof, DNA, or any combination thereof.

8. The method of claim 7, wherein the metal oxide comprises iron oxide.

9. The method of claim 7, wherein the polysaccharide or derivative thereof comprises zymosan, lipopolysaccharide, or any combination thereof.

10. A phagocytic cell comprising loaded polymeric microparticles made by the method claim 1 in the cytosol of the phagocytic cell.

11. The phagocytic cell of claim 10, wherein the phagocytic cell comprises a macrophage, a dendric cell, a neutrophil, a monocytes, a mast cell, or a non-professional phagocytic cell.

12. The phagocytic cell of claim 11, wherein the non-professional phagocytic cell comprises an epithelial cell or a fibroblast.

13. A method for treating a disease in a subject, the method comprising administering the phagocytic cell according to claim 10 to the subject.

14. The method of claim 13, wherein the phagocytic cell or the composition is administered to the subject intravenously.

15. The method of claim 13, wherein the disease comprises cancer, rheumatoid arthritis, atherosclerosis, Alzheimer's disease, multiple sclerosis, obesity, or another disease characterized by chronic local inflammation.

16. The method of claim 15, wherein the cancer comprises non-Hodgkins lymphoma, neuroblastoma, sarcoma, metastatic brain cancers, ovarian cancer, prostate cancer, breast cancer, lymphoma, non-small cell lung carcinoma, gastric cancer, gastroesophageal junction adenocarcinoma, melanoma, squamous cell carcinoma, pancreatic cancer, hepatocellular carcinoma, colorectal cancer, angiosarcoma, head and neck cancer, ovarian cancer, solid tumors, multiple myeloma, glioblastoma, testicular cancer, urothelial cancer, adenocortical carcinoma, clear cell renal cell carcinoma, small cell lung renal cell carcinoma, nasopharyngeal cancer, glioma, gall bladder cancer, thyroid tumor, bone cancer, cervical cancer, uterine cancer, endometrial cancer, vulvar cancer, bladder cancer, colon cancer, colorectal cancer, pancreatic cancer, neuronal cancers, mesothelioma, cholangiocarcinoma, small bowel adenocarcinoma, epidermoid carcinoma, cancer of the pleural or peritoneal membranes, another cancer, or any combination thereof.

17. The method of claim 13, further comprising administering an additional treatment to the subject.

18. The method of claim 17, wherein the additional treatment comprises radiation, chemotherapy, immunotherapy, bone marrow transplant, hormone therapy, surgery, or any combination thereof.

19. The method of claim 13, further comprising isolating the phagocytic cells from the subject prior to incubating the phagocytic cells with the loaded polymeric microparticles.

20. The method of claim 13, wherein the subject is a mammal or a bird.
